(19) United States
(12) Patent Application Publication
(10) Pub. No.: US 2005/0176011 A1
(43) Pub. Date: Aug. 11, 2005

(54) Identification of biomarkers for detecting prostate cancer

(76) Inventors: Daniel W. Chan, Clarksville, MD (US); Jinong Li, Ellicott City, MD (US); Zhen Zhang, Dayton, MD (US)

Correspondence Address: EDWARDS & ANGELL, LLP, P.O. BOX 55874, BOSTON, MA, 02205, US

(21) Appl. No.: 10/512,731

(22) PCT Filed: Apr. 25, 2003
(86) PCT No.: PCT/US03/12729

Related U.S. Application Data
(60) Provisional application No. 60/375,719, filed on Apr. 26, 2002.

(51) Int. Cl. C12Q 1/68; G01N 33/574; G01N 33/00

(52) U.S. Cl. 435/6; 435/7.23; 436/86

(57) ABSTRACT

The present invention relates to a method of qualifying prostate cancer status in a subject comprising: (a) measuring at least one of the disclosed biomarkers in a sample from the subject and (b) correlating the measurement with prostate cancer status. The invention further relates to kits for qualifying prostate cancer status in a subject.

The present application claims the benefit of U.S. provisional application No. 60/375,719, filed Apr. 26, 2002 which is incorporated herein, by reference, in its entirety.

### FIELD OF THE INVENTION

The invention provides for the reliable detection and identification of biomarkers, important for the diagnosis and prognosis of prostate cancer. The plasma protein profile in prostate cancer patients are distinguished from non-neoplastic individuals using SELDI analysis. This technique provides a simple yet sensitive approach to diagnose prostate cancer using serum or plasma samples.

### BACKGROUND OF THE INVENTION

Prostate carcinoma is the most common type of cancer in men. In the year 2002, it is expected to account for 189,000 new cancer cases among men and 30,200 will die from this disease. Early detection of prostate cancer when the cancer is confined to the prostate gland has the best chance of cure through radical prostatectomy (surgery). Although PSA is considered as an effective tumor marker and is for all intents and purposes organ specific, it is not cancer specific. There is considerable overlap in PSA concentrations in men with prostate cancer and men with benign prostatic diseases. PSA could not differentiate men with organ confined prostate cancer (who would benefit from surgery) from those men with non-organ confined prostate cancer (who would not benefit from surgery). Therefore, PSA is not effective in selecting patients for radical prostatectomy.

Early detection and diagnosis of prostate cancer currently relies on digital rectal examinations (DRE), prostate specific antigen (PSA) measurements, transrectal ultrasonography (TRUS), and transrectal needle biopsy (TRNB). At present, serum PSA measurement in combination with DRE represents the leading tool used to detect and diagnose prostate cancer.

Commercially-available PSA assays are commonly sold as kits, and the assays performed in regional or local laboratories. However, prostate specific antigen (PSA) and prostatic acid phosphatase (PAP), have limited therapeutic and diagnostic potential. For example, PSA levels do not always correlate well with the presence of prostate cancer, being positive in a percentage of non-prostate cancer cases, including benign prostatic hyperplasia (BPH). Furthermore, PSA measurements correlate with prostate volume, and do not indicate the level of metastasis.

These kits play a part in the current strategy for early detection of prostate cancer. A problem arises, however, when a modestly abnormal PSA value (4-10 ng/ml) is encountered in the context of a negative digital rectal exam (DRE). Only 20-30% of individuals with such findings will demonstrate carcinoma on biopsy (Kantoff and Talcott, 8(3) Hematol. Oncol. Clinics N Amer 555 (1994)). It has therefore been important to develop strategies that increase the positive predictive value of PSA testing. Such strategies now include establishing age-adjusted normal ranges, determining the free to total PSA ratio, correcting for prostate gland mass (density), and calculating the rate of change of PSA values (Kantoff and Talcott, 8(3) Hematol. Oncol Clinics N Amer 555 (1994) and Brawer, 45 CA-A Cancer J Clinicians 148 (1995)). While each of these strategies has made a contribution, considerable uncertainty nevertheless remains about how to proceed with a patient who is PSA positive and DRE negative.

In addition, PSA is not a disease-specific marker, as elevated levels of PSA are detectable in a large percentage of patients with BPH and prostatitis (25-86%) (Gao et al., 1997, Prostate 31: 264-281), as well as in other nonmalignant disorders and in some normal men, a factor which significantly limits the diagnostic specificity of this marker. For example, elevations in serum PSA of between 4 to 10 ng/ml are observed in BPH, and even higher values are observed in prostatitis, particularly acute prostatitis. BPH is an extremely common condition in men. Further confusing the situation is the fact that serum PSA elevations may be observed without any indication of disease from DRE, and vice-versa. Moreover, it is now recognized that PSA is not prostate-specific (Gao et al., supra, for review).

There is also a need for more reliable and informative staging and prognostic methods in the management of advanced prostate cancer. Clinically staging prostate tumors relies on rectal examination to determine whether the tumor remains within the borders of the prostatic capsule (locally confined) or extends beyond it (locally advanced), in combination with serum PSA determinations and transrectal ultrasound guided biopsies. However, none of these techniques has proven reliable for predicting progression of the disease.

A need therefore, exists which can specifically identify prostate cancer, can distinguish prostate cancer from benign hyperplasia, can identify prostate cancer even though PSA levels are low, and identify the stages of disease progression.

### SUMMARY OF THE INVENTION

The present invention provides, for the first time, novel protein markers that are differentially present in the samples of human cancer patients and in the samples of control subjects. The present invention also provides sensitive and quick methods and kits that can be used as an aid for diagnosis of human cancer by detecting these novel markers. The measurement of these markers, alone or in combination, in patient samples provides information that diagnostician can correlate with a probable diagnosis of human cancer or a negative diagnosis (e.g., normal or disease-free). All the markers are characterized by molecular weight. The markers can be resolved from other proteins in a sample by using a variety of fractionation techniques, e.g., chromatographic separation coupled with mass spectrometry, or by traditional immunoassays.

In preferred embodiments, the method of resolution involves Surface-Enhanced Laser Desorption/Ionization (“SELDI”) mass spectrometry, in which the surface of the mass spectrometry probe comprises adsorbents that bind the markers.

In other preferred embodiments, comparative protein profiles are generated using the ProteinChip Biomarker System from patients diagnosed with prostate cancer and from patients without known neoplastic diseases. A subset of biomarkers was selected based on collaborative results from supervised analytical methods. Preferred analytical methods include the Classification And Regression Tree (CART), implemented in Biomarker Pattern Software V4.0 (BPS) (Ciphergen, Calif.), and the Unified Maximum Separability Analysis (UMSA) procedure, implemented in ProPeak (3Z Informatics, SC).

In a preferred embodiment, the analytical methods are used individually and in cross-comparison to screen for peaks that are most contributory towards the discrimination between non cancer diseases of the prostate; organ confined prostate cancer; non-organ confined prostate cancer; pre-invasive stages of prostate cancer; malignant versus benign forms of cancer; different cancer stages of prostate cancer; and the non-cancer controls.

In another aspect, the biomarkers were purified and identified. The selected biomarkers, are evaluated individually and in combination through multivariate logistic regression. The biomarkers are also evaluated together with known tumor markers such as, for example, prostate specific antigen (PSA) or prostatic acid phosphatase (PAP).

While the absolute identity of these markers is not yet known, such knowledge is not necessary to measure them in a patient sample, because they are sufficiently characterized by, e.g., mass and by affinity characteristics. It is noted that molecular weight and binding properties are characteristic properties of these markers and not limitations on means of detection or isolation. Furthermore, using the methods described herein or other methods known in the art, the absolute identity of the markers can be determined.

The present invention also relates to biomarkers designated as Markers I through XXXII. Protein markers of the invention can be characterized in one or more of several respects. In particular, in one aspect, these markers are characterized by molecular weights under the conditions specified herein, particularly as determined by mass spectral analysis. In another aspect, the markers can be characterized by features of the markers' mass spectral signature such as size (including area) and/or shape of the markers' spectral peaks, features including proximity, size and shape of neighboring peaks, etc. In yet another aspect, the markers can be characterized by affinity binding characteristics, particularly ability to binding to an IMAC nickel adsorbent under specified conditions, however, other metals, e.g., copper, may also be used. In preferred embodiments, markers of the invention may be characterized by each of such aspects, i.e. molecular weight, mass spectral signature and IMAC3-Ni²⁺ absorbent binding.

For the mass values of the markers disclosed herein, the mass accuracy of the spectral instrument is considered to be about within +/− 0.15 percent of the disclosed molecular weight value. Additionally, to such recognized accuracy variations of the instrument, the spectral mass determination can vary within resolution limits of from about 400 to 1000 m/dm, where m is mass and dm is the mass spectral peak width at 0.5 peak height. Those mass accuracy and resolution variances associated with the mass spectral instrument and operation thereof are reflected in the use of the term “about” in the disclosure of the mass of each of Markers I through XXXII. It is also intended that such mass accuracy and resolution variances and thus meaning of the term “about” with respect to the mass of each of the markers disclosed herein is inclusive of variants of the markers as may exist due to sex, genotype and/or ethnicity of the subject and the particular cancer or origin or stage thereof.

Molecular weights as measured by mass spectrometry are specified for each marker as follows:
Marker I: having a molecular weight of about 7.808 kD
Marker II: having a molecular weight of about 14.576 kD
Marker III: having a molecular weight of about 2.062 kD
Marker IV: having a molecular weight of about 7.974 kD
Marker V: having a molecular weight of about 6.677 kD
Marker VI: having a molecular weight of about 3.936 kD
Marker VII: having a molecular weight of about 60.958 kD
Marker VIII: having a molecular weight of about 5.149 kD
Marker IX: having a molecular weight of about 5.861 kD
Marker X: having a molecular weight of about 28.098 kD
Marker XI: having a molecular weight of about 2.996 kD
Marker XII: having a molecular weight of about 24.346 kD
Marker XIII: having a molecular weight of about 6.722 kD
Marker XIV: having a molecular weight of about 5.999 kD
Marker XV: having a molecular weight of about 6.158 kD
Marker XVI: having a molecular weight of about 55.785 kD
Marker XVII: having a molecular weight of about 2.540 kD
Marker XVIII: having a molecular weight of about 8.019 kD
Marker XIX: having a molecular weight of about 4.658 kD
Marker XX: having a molecular weight of about 14.703 kD
Marker XXI: having a molecular weight of about 2.68 kD
Marker XXII: having a molecular weight of about 3.16 kD
Marker XXIII: having a molecular weight of about 10.3 kD
Marker XXIV: having a molecular weight of about 10.8 kD
Marker XXV: having a molecular weight of about 12.7 kD
Marker XXVI: having a molecular weight of about 17.9 kD
Marker XXVII: having a molecular weight of about 2.79 kD
Marker XXVIII: having a molecular weight of about 3.32 kD
Marker XXIX: having a molecular weight of about 4.29 kD
Marker XXX: having a molecular weight of about 15.9 kD
Marker XXXI: having a molecular weight of about 16.1 kD
Marker XXXII: having a molecular weight of about 16.3 kD

Markers I through XXXII also are characterized by their mass spectral signature. The mass spectra of each of Markers I through XXXII are set forth in FIGS. 1-5.

Each of Markers Markers I through XXXII also is characterized by its ability to bind to an IMAC3-Ni²⁺ adsorbent, as specified herein.

Preferred methods for detection and diagnosis of cancer comprise detecting at least one or more protein biomarkers in a subject sample, and; correlating the detection of one or more protein biomarkers with a diagnosis of cancer, wherein the correlation takes into account the detection of one or more biomarker in each diagnosis, as compared to normal subjects, wherein the one or more protein markers are selected from:
Marker I: having a molecular weight of about 7.808 kD
Marker II: having a molecular weight of about 14.576 kD
Marker III: having a molecular weight of about 2.062 kD
Marker IV: having a molecular weight of about 7.974 kD
Marker V: having a molecular weight of about 6.677 kD
Marker VI: having a molecular weight of about 3.936 kD
Marker VII: having a molecular weight of about 60.958 kD
Marker VIII: having a molecular weight of about 5.149 kD
Marker IX: having a molecular weight of about 5.861 kD
Marker X: having a molecular weight of about 28.098 kD
Marker XI: having a molecular weight of about 2.996 kD
Marker XII: having a molecular weight of about 24.346 kD
Marker XIII: having a molecular weight of about 6.722 kD
Marker XIV: having a molecular weight of about 5.999 kD
Marker XV: having a molecular weight of about 6.158 kD
Marker XVI: having a molecular weight of about 55.785 kD
Marker XVII: having a molecular weight of about 2.540 kD
Marker XVIII: having a molecular weight of about 8.019 kD
Marker XIX: having a molecular weight of about 4.658 kD
Marker XX: having a molecular weight of about 14.703 kD
Marker XXI: having a molecular weight of about 2.68 kD
Marker XXII: having a molecular weight of about 3.16 kD
Marker XXIII: having a molecular weight of about 10.3 kD
Marker XXIV: having a molecular weight of about 10.8 kD
Marker XXV: having a molecular weight of about 12.7 kD
Marker XXVI: having a molecular weight of about 17.9 kD
Marker XXVII: having a molecular weight of about 2.79 kD
Marker XXVIII: having a molecular weight of about 3.32 kD
Marker XXIX: having a molecular weight of about 4.29 kD
Marker XXX: having a molecular weight of about 15.9 kD
Marker XXXI: having a molecular weight of about 16.1 kD
Marker XXXII: having a molecular weight of about 16.3 kD.

In a preferred embodiment, the present invention provides for a method for detecting, diagnosing and differentiating between pre-invasive, benign, malignant or different malignant stages of cancer, wherein the method comprises using a biochip array for detecting at least one biomarker in a subject sample; evaluating at least one biomarker in a subject sample, and correlating the detection of one or more protein biomarkers with cancer.

Preferably, one or more protein biomarkers are used for detecting, diagnosing and differentiating between pre-invasive, benign, malignant or different malignant stages of cancer; wherein, the one or more protein markers are selected from:
Marker XVII: having a molecular weight of about 2.540 kD
Marker XVIII: having a molecular weight of about 8.018 kD
Marker XIX: having a molecular weight of about 4.658kD, and
Marker XX: having a molecular weight of about 14.703 kD.

An additional group of markers used for detecting, diagnosing and differentiating between pre-invasive, benign, malignant or different malignant stages of cancer; wherein, the one or more protein markers are selected from:
Marker XXVII: having a molecular weight of about 2.79 kD
Marker XXVIII: having a molecular weight of about 3.32 kD
Marker XXIX: having a molecular weight of about 4.29 kD
Marker XXX: having a molecular weight of about 15.9 kD
Marker XXXI: having a molecular weight of about 16.1 kD
Marker XXXII: having a molecular weight of about 16.3 kD.

In a preferred embodiment, the invention provides for differentiating between a diagnosis of prostate cancer and non-prostate cancer comprising:
(a) detecting in a subject sample an amount of at least one biomarker selected from the group consisting of:
   Marker I: having a molecular weight of about 7.808 kD
   Marker II: having a molecular weight of about 14.576 kD
   Marker III: having a molecular weight of about 2.061 kD
   Marker IV: having a molecular weight of about 7.973 kD
   Marker V: having a molecular weight of about 6.677 kD and
   Marker VI: having a molecular weight of about 3.935 kD; and
(b) correlating the amount with a diagnosis of prostate cancer or non-prostate cancer.

In another aspect of the invention, at least one biomarker is used to diagnose prostate cancer. Preferably, a plurality of the biomarkers are detected.

In one aspect of the invention, a single biomarker is used in combination with one or more cancer antigens for diagnosing cancer. However, a plurality of the markers can be used in combination with one or more cancer antigens for diagnosing cancer. For example, cancer antigens, include but not limited to prostate specific antigen (PSA) or prostatic acid phosphatase (PAP).

In a most preferred embodiment, the biomarkers are able to detect, diagnose and differentiate between diseases of the prostate and/or benign prostate cancer, pre-invasive or different malignant stages of prostate cancer, when antigens, such as PSA, are detected at less than about 3 ng/ml or are undetectable by methods used in the art.

In another preferred embodiment, the invention provides for a diagnosis of organ defined prostate cancer and non-organ-defined prostate cancer comprising:
(a) detecting in a subject sample an amount of at least one biomarker selected from the group consisting of:
   Marker XVII: having a molecular weight of about 2.540 kD
   Marker XVIII: having a molecular weight of about 8.018 kD
   Marker XIX: having a molecular weight of about 4.658kD,
   Marker XX: having a molecular weight of about 14.703 kD;
      and
(b) correlating the amount with a diagnosis of organ-defined prostate cancer or non-organ-defined prostate cancer.

In another preferred embodiment, the invention provides for a diagnosis of organ defined prostate cancer and non-organ-defined prostate cancer comprising:
(a) detecting in a subject sample an amount of at least one biomarker selected from the group consisting of:
Marker XXVII: having a molecular weight of about 2.79 kD
Marker XXVIII: having a molecular weight of about 3.32 kD
Marker XXIX: having a molecular weight of about 4.29 kD
Marker XXX: having a molecular weight of about 15.9 kD
Marker XXXI: having a molecular weight of about 16.1 kD
Marker XXXII: having a molecular weight of about 16.3 kD.
(b) correlating the amount with a diagnosis of organ-defined prostate cancer or non-organ-defined prostate cancer.

In a preferred embodiment, at least one biomarker is used to differentiate between benign versus malignant prostate cancer and to determine whether the prostate cancer is confined to the prostate or has metastasized to other organs. A single biomarker can be used in combination with one or more prostate cancer antigens for diagnosing prostate cancer, for example, cancer antigens, include but not limited to prostate specific antigen (PSA) or prostatic acid phosphatase (PAP). Most preferred is the detection of the biomarkers of the invention when the known prostate cancer antigens are found in amounts wherein, a diagnosis using the results of a known marker, are not definitive. For example, when PSA is detected at levels of about 3 ng/ml.

In another preferred embodiment, one or more protein biomarkers are used in diagnosing and differentiating between pre-invasive or the different malignant stages of cancer; wherein, the one or more protein markers are selected from:
Marker I: having a molecular weight of about 7.808 kD
Marker II: having a molecular weight of about 14.576 kD
Marker III: having a molecular weight of about 2.062 kD
Marker IV: having a molecular weight of about 7.974 kD
Marker V: having a molecular weight of about 6.677 kD
Marker VI: having a molecular weight of about 3.936 kD
Marker VII: having a molecular weight of about 60.958 kD
Marker VIII: having a molecular weight of about 5.149 kD; and
correlating the detection of one or more protein biomarkers with a diagnosis of a pre-invasive or different malignant stages of prostate cancer. The correlation can take into account the detection of one or more protein biomarkers in each diagnosis, as compared to normal subjects.

Preferably, one or more protein biomarkers are used to determine whether the prostate cancer is at a pre-invasive stage or to identify the different malignant stages of prostate cancer. Also preferred is a detection of a plurality of the biomarkers, wherein at least about two biomarkers are detected.

In another preferred embodiment, a single biomarker is used in combination with one or more prostate cancer antigens for determining whether the prostate cancer is at a pre-invasive stage or to identify the different malignant stages of prostate cancer.

In another aspect of the invention, a plurality of the markers are used in combination with one or more prostate cancer antigens, for example, PSA, for determining whether the prostate cancer is at a pre-invasive stage or to identify the different malignant stages of prostate cancer.

In a preferred embodiment, the detection of biomarkers determine whether prostate cancer is at a pre-invasive stage or can determine the different stages of prostate cancer progression when levels of PSA are not detected at levels to make a definitive diagnosis, for example, when PSA is detected at levels that are less than about 3 ng/ml.

In a preferred embodiment, one or more protein biomarkers are used to differentiate between prostate cancer and different diseases of the prostate, even though levels of known markers, such as PSA, are below definitive diagnostic levels, such as when PSA is detected at levels less than about 3 ng/ml. Diseases of the prostate, include, but are not limited to benign prostatic hyperplasia (BPH), prostatitis, prostatic intraepithelial neoplasia (PIN) and cancer. Examples of prostate cancer, include, but are not limited to, adenocarcinoma, small cell undifferentiated carcinoma and mucinous (colloid) cancer.

The accuracy of a diagnostic test is characterized by a Receiver Operating Characteristic curve (“ROC curve”). An ROC is a plot of the true positive rate against the false positive rate for the different possible cutpoints of a diagnostic test. An ROC curve shows the relationship between sensitivity and specificity. That is, an increase in sensitivity will be accompanied by a decrease in specificity. The closer the curve follows the left axis and then the top edge of the ROC space, the more accurate the test. Conversely, the closer the curve comes to the 45-degree diagonal of the ROC graph, the less accurate the test. The area under the ROC is a measure of test accuracy. The accuracy of the test depends on how well the test separates the group being tested into those with and without the disease in question. An area under the curve (referred to as “AUC”) of 1 represents a perfect test, while an area of 0.5 represents a less useful test. Thus, preferred biomarkers and diagnostic methods of the present invention have an AUC greater than 0.50, more preferred tests have an AUC greater than 0.60, more preferred tests have an AUC greater than 0.70.

Preferably, the biomarkers of the invention are detected in samples of blood, blood plasma, serum, urine, tissue, cells, organs and seminal fluids.

Preferred detection methods include use of a biochip array. Biochip arrays useful in the invention include protein and nucleic acid arrays. One or more markers are captured on the biochip array and subjected to laser ionization to detect the molecular weight of the markers. Analysis of the markers is, for example, by molecular weight of the one or more markers against a threshold intensity that is normalized against total ion current. Preferably, logarithmic transformation is used for reducing peak intensity ranges to limit the number of markers detected.

In preferred methods of the present invention, the step of correlating the measurement of the biomarkers with prostate cancer status is performed by a software classification algorithm. Preferably, data is generated on immobilized subject samples on a biochip array, by subjecting said biochip array to laser ionization and detecting intensity of signal for mass/charge ratio; and, transforming the data into computer readable form; and executing an algorithm that classifies the data according to user input parameters, for detecting signals that represent markers present in prostate cancer patients and are lacking in non-cancer subject controls.

Preferably the biochip surfaces are, for example, ionic, anionic, comprised of immobilized nickel ions, comprised of a mixture of positive and negative ions, comprised of one or more antibodies, single or double stranded nucleic acids, proteins, peptides or fragments thereof, amino acid probes, or phage display libraries.

In other preferred methods one or more of the markers are measured using laser desorption/ionization mass spectrometry, comprising providing a probe adapted for use with a mass spectrometer comprising an adsorbent attached thereto, and contacting the subject sample with the adsorbent, and; desorbing and ionizing the marker or markers from the probe and detecting the deionized/ionized markers with the mass spectrometer.

Preferably, the laser desorption/ionization mass spectrometry comprises: providing a substrate comprising an adsorbent attached thereto; contacting the subject sample with the adsorbent; placing the substrate on a probe adapted for use with a mass spectrometer comprising an adsorbent attached thereto; and, desorbing and ionizing the marker or markers from the probe and detecting the desorbed/ionized marker or markers with the mass spectrometer.

The adsorbent can for example be, hydrophobic, hydrophilic, ionic or metal chelate adsorbent, such as, nickel or an antibody, single- or double stranded oligonucleotide, amino acid, protein, peptide or fragments thereof.

In another embodiment, a process for purification of a biomarker, comprising fractioning a sample comprising one or more protein biomarkers by size-exclusion chromatography and collecting a fraction that includes the one or more biomarker; and/or fractionating a sample comprising the one or more biomarkers by anion exchange chromatography and collecting a fraction that includes the one or more biomarkers. Fractionation is monitored for purity on normal phase and immobilized nickel arrays. Generating data on immobilized marker fractions on an array, is accomplished by subjecting said array to laser ionization and detecting intensity of signal for mass/charge ratio; and, transforming the data into computer readable form; and executing an algorithm that classifies the data according to user input parameters, for detecting signals that represent markers present in cancer patients and are lacking in non-cancer subject controls. Preferably fractions are subjected to gel electrophoresis and correlated with data generated by mass spectrometry. In one aspect, gel bands representative of potential markers are excised and subjected to enzymatic treatment and are applied to biochip arrays for peptide mapping.

In another aspect one or more biomarkers are selected from gel bands representing:
Marker I: having a molecular weight of about 7.808 kD
Marker II: having a molecular weight of about 14.576 kD
Marker III: having a molecular weight of about 2.062 kD
Marker IV: having a molecular weight of about 7.974 kD
Marker V: having a molecular weight of about 6.677 kD
Marker VI: having a molecular weight of about 3.936 kD
Marker VII: having a molecular weight of about 60.958 kD
Marker VIII: having a molecular weight of about 5.149 kD
Marker IX: having a molecular weight of about 5.861 kD
Marker X: having a molecular weight of about 28.098 kD
Marker XI: having a molecular weight of about 2.996 kD
Marker XII: having a molecular weight of about 24.346 kD
Marker XIII: having a molecular weight of about 6.722 kD
Marker XIV: having a molecular weight of about 5.999 kD
Marker XV: having a molecular weight of about 6.158 kD
Marker XVI: having a molecular weight of about 55.785 kD
Marker XVII: having a molecular weight of about 2.540 kD
Marker XVIII: having a molecular weight of about 8.019 kD
Marker XIX: having a molecular weight of about 4.658
Marker XX: having a molecular weight of about 14.703 kD
Marker XXI: having a molecular weight of about 2.68 kD
Marker XXII: having a molecular weight of about 3.16 kD
Marker XXIII: having a molecular weight of about 10.3 kD
Marker XXIV: having a molecular weight of about 10.8 kD
Marker XXV: having a molecular weight of about 12.7 kD
Marker XXVI: having a molecular weight of about 17.9 kD
Marker XXVII: having a molecular weight of about 2.79 kD
Marker XXVIII: having a molecular weight of about 3.32 kD
Marker XXIX: having a molecular weight of about 4.29 kD
Marker XXX: having a molecular weight of about 15.9 kD
Marker XXXI: having a molecular weight of about 16.1 kD
Marker XXXII: having a molecular weight of about 16.3 kD.

Purified proteins for screening and aiding in the diagnosis of prostate cancer and/or generation of antibodies for further diagnostic assays are provided for. Purified proteins are selected from:
Marker I: having a molecular weight of about 7.808 kD
Marker II: having a molecular weight of about 14.576 kD
Marker III: having a molecular weight of about 2.062 kD
Marker IV: having a molecular weight of about 7.974 kD
Marker V: having a molecular weight of about 6.677 kD
Marker VI: having a molecular weight of about 3.936 kD
Marker VII: having a molecular weight of about 60.958 kD
Marker VIII: having a molecular weight of about 5.149 kD
Marker IX: having a molecular weight of about 5.861 kD
Marker X: having a molecular weight of about 28.098 kD
Marker XI: having a molecular weight of about 2.996 kD
Marker XII: having a molecular weight of about 24.346 kD
Marker XIII: having a molecular weight of about 6.722 kD
Marker XIV: having a molecular weight of about 5.999 kD
Marker XV: having a molecular weight of about 6.158 kD
Marker XVI: having a molecular weight of about 55.785 kD
Marker XVII: having a molecular weight of about 2.540 kD
Marker XVIII: having a molecular weight of about 8.019 kD
Marker XIX: having a molecular weight of about 4.658 kD
Marker XX: having a molecular weight of about 14.703 kD
Marker XXI: having a molecular weight of about 2.68 kD
Marker XXII: having a molecular weight of about 3.16 kD
Marker XXIII: having a molecular weight of about 10.3 kD
Marker XXIV: having a molecular weight of about 10.8 kD
Marker XXV: having a molecular weight of about 12.7 kD
Marker XXVI: having a molecular weight of about 17.9 kD
Marker XXVII: having a molecular weight of about 2.79 kD
Marker XXVIII: having a molecular weight of about 3.32 kD
Marker XXIX: having a molecular weight of about 4.29 kD
Marker XXX: having a molecular weight of about 15.9 kD
Marker XXXI: having a molecular weight of about 16.1 kD
Marker XXXII: having a molecular weight of about 16.3 kD.

The invention further provides for kits for aiding the diagnosis of cancer, comprising:
an adsorbent attached to a substrate, wherein the adsorbent retains one or more biomarker selected from:
   Marker I: having a molecular weight of about 7.808 kD
   Marker II: having a molecular weight of about 14.576 kD
   Marker III: having a molecular weight of about 2.062 kD
   Marker IV: having a molecular weight of about 7.974 kD
   Marker V: having a molecular weight of about 6.677 kD
   Marker VI: having a molecular weight of about 3.936 kD
   Marker VII: having a molecular weight of about 60.958 kD
   Marker VIII: having a molecular weight of about 5.149 kD
   Marker IX: having a molecular weight of about 5.861 kD
   Marker X: having a molecular weight of about 28.098 kD
   Marker XI: having a molecular weight of about 2.996 kD
   Marker XII: having a molecular weight of about 24.346 kD
   Marker XIII: having a molecular weight of about 6.722 kD
   Marker XIV: having a molecular weight of about 5.999 kD
   Marker XV: having a molecular weight of about 6.158 kD
   Marker XVI: having a molecular weight of about 55.785 kD
   Marker XVII: having a molecular weight of about 2.540 kD
   Marker XVIII: having a molecular weight of about 8.019 kD
   Marker XIX: having a molecular weight of about 4.658 kD
   Marker XX: having a molecular weight of about 14.703 kD
   Marker XXI: having a molecular weight of about 2.68 kD
   Marker XXII: having a molecular weight of about 3.16 kD
   Marker XXIII: having a molecular weight of about 10.3 kD
   Marker XXIV: having a molecular weight of about 10.8 kD
   Marker XXV: having a molecular weight of about 12.7 kD
   Marker XXVI: having a molecular weight of about 17.9 kD
   Marker XXVII: having a molecular weight of about 2.79 kD
   Marker XXVIII: having a molecular weight of about 3.32 kD
   Marker XXIX: having a molecular weight of about 4.29 kD
   Marker XXX: having a molecular weight of about 15.9 kD
   Marker XXXI: having a molecular weight of about 16.1 kD
   Marker XXXII: having a molecular weight of about 16.3 kD.

Preferably, the kit comprises written instructions for use of the kit for detection of cancer and the instructions provide for contacting a test sample with the absorbent and detecting one or more biomarkers retained by the adsorbent.

The kit provides for a substrate which allows for adsorption of said adsorbent. Preferably, the substrate can be hydrophobic, hydrophilic, charged, polar, metal ions.

The kit also provides for an adsorbent wherein the adsorbent is an antibody, single or double stranded oligonucleotide, amino acid, protein, peptide or fragments thereof.

Detection of one or more protein biomarkers using the kit, is by mass spectrometry or immunoassays such as an ELISA.

In another preferred embodiment biomarkers, purified on a biochip and identified by their molecular weights, are selected from:
Marker I: having a molecular weight of about 7.808 kD
Marker II: having a molecular weight of about 14.576 kD
Marker III: having a molecular weight of about 2.062 kD
Marker IV: having a molecular weight of about 7.974 kD
Marker V: having a molecular weight of about 6.667 kD
Marker VI: having a molecular weight of about 3.936 kD
Marker VII: having a molecular weight of about 60.958 kD
Marker VIII: having a molecular weight of about 5.149 kD
Marker IX: having a molecular weight of about 5.861 kD
Marker X: having a molecular weight of about 28.098 kD
Marker XI: having a molecular weight of about 2.996 kD
Marker XII: having a molecular weight of about 24.346 kD
Marker XIII: having a molecular weight of about 6.722 kD
Marker XIV: having a molecular weight of about 5.999 kD
Marker XV: having a molecular weight of about 6.159 kD
Marker XVI: having a molecular weight of about 55.784 kD
Marker XVII: having a molecular weight of about 2.540 kD
Marker XVIII: having a molecular weight of about 8.019 kD
Marker XIX: having a molecular weight of about 4.658 kD
Marker XX: having a molecular weight of about 14.703 kD
Marker XXI: having a molecular weight of about 2.68 kD
Marker XXII: having a molecular weight of about 3.16 kD
Marker XXIII: having a molecular weight of about 10.3 kD
Marker XXIV: having a molecular weight of about 10.8 kD
Marker XXV: having a molecular weight of about 12.7 kD
Marker XXVI: having a molecular weight of about 17.9 kD
Marker XXVII: having a molecular weight of about 2.79 kD
Marker XXVIII: having a molecular weight of about 3.32 kD
Marker XXIX: having a molecular weight of about 4.29 kD
Marker XXX: having a molecular weight of about 15.9 kD
Marker XXXI: having a molecular weight of about 16.1 kD
Marker XXXII: having a molecular weight of about 16.3 kD

In another preferred embodiment, at least two purified biomarkers comprise a composition of a combination of any of the Markers I through XXXII for use in differentiating between diseases of the prostate, prostate cancer, and the different stages of prostate cancer.

Preferably each of the markers in the compositions are purified.

Other aspects of the invention are described infra.

### BRIEF DESCRIPTION OF THE FIGURES

FIG. 1A-F shows mass spectra of Markers for Panel 1. In those Figures, the mass spectral peak of the specified marker is designated within the depicted spectra with an arrow. The Figure designation is next to each of the referred to spectra.

FIG. 2A-J shows mass spectra of Markers for Panel 2. In those Figures, the mass spectral peak of the specified marker is designated within the depicted spectra with an arrow. The Figure designation is set next to each of the referred to spectra.

FIG. 3A-D shows mass spectra of Markers for Panel 3. In those Figures, the mass spectral peak of the specified marker is designated within the depicted spectra with an arrow. The Figure designation is set next to each of the referred to spectra.

FIG. 4A-F shows mass spectra of additional markers for Panel 2. In those Figures, the mass spectral peak of the specified marker is designated within the depicted spectra with an arrow. The Figure designation is set above each of the referred to spectra.

FIG. 5A-F shows mass spectra of additional markers for Panel 3. In those Figures, the mass spectral peak of the specified marker is designated within the depicted spectra with an arrow. The Figure designation is set above each of the referred to spectra.

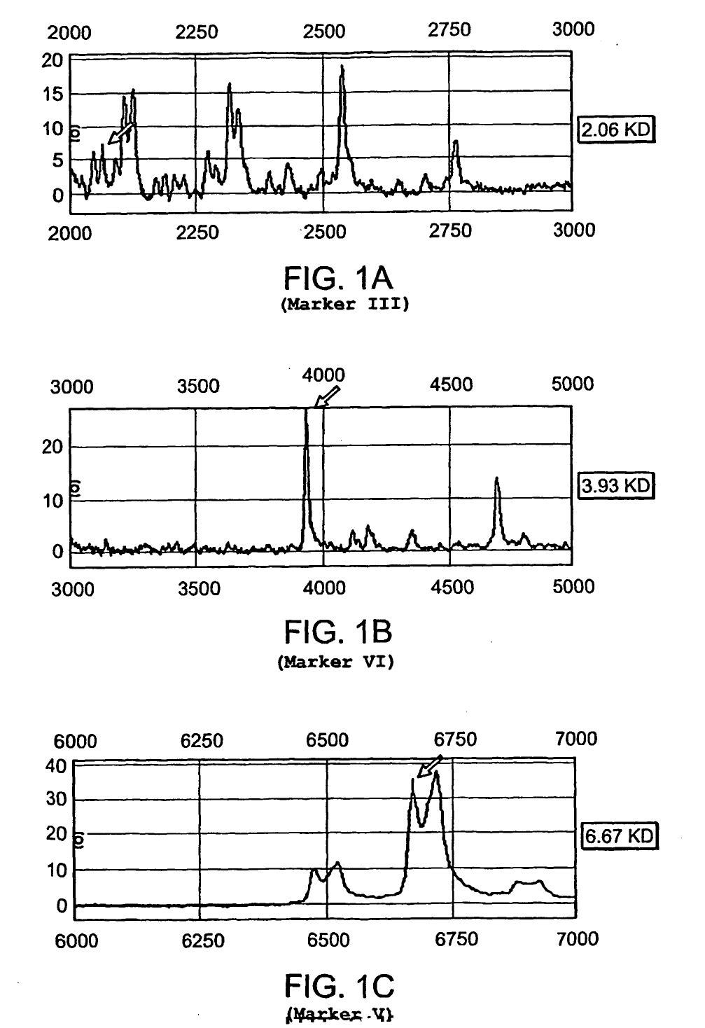

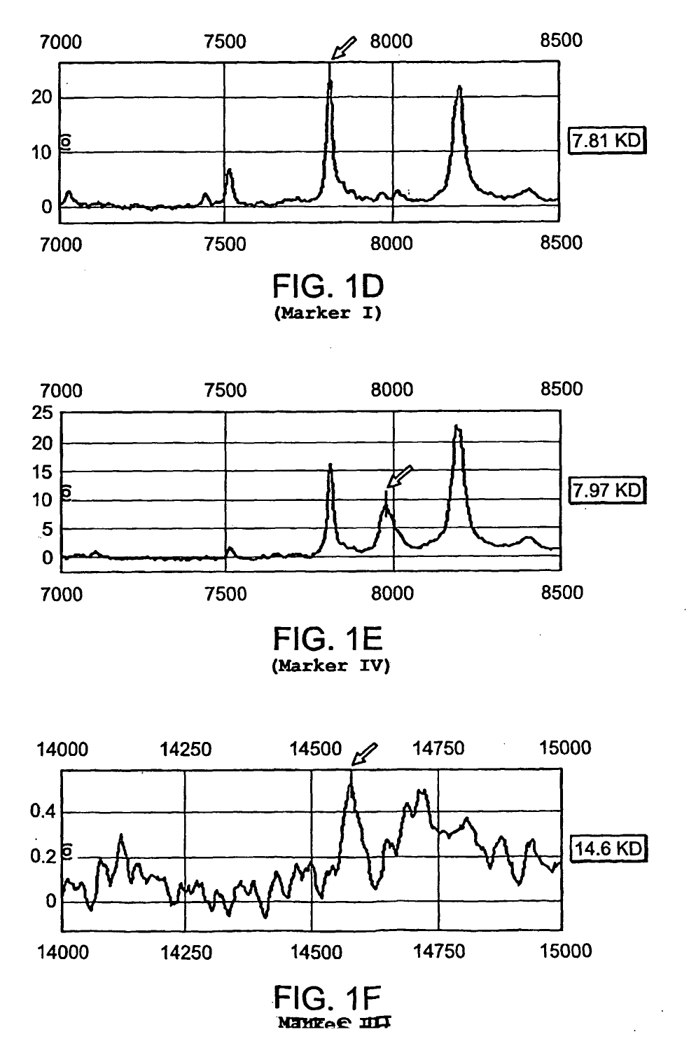

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, “diseases of the prostate” or “prostate disease,” or “condition of the prostate,” as used herein, refer to any disease or condition of the prostate including, but not limited to, benign prostatic hyperplasia (BPH), prostatitis, prostatic intraepithelial neoplasia (PIN) and cancer.

As used herein, “prostate cancer,” as used herein, refers to any malignant disease of the prostate including, but not limited to, adenocarcinoma, small cell undifferentiated carcinoma and mucinous (colloid) cancer.

As used herein, the terms “metastatic prostate cancer” and “metastatic disease” mean prostate cancers which have spread to regional lymph nodes or to distant sites, and are meant to include stage D disease under the AUA system and stage T×N×M+ under the TNM system. As is the case with locally advanced prostate cancer, surgery is generally not indicated for patients with metastatic disease, and hormonal (androgen ablation) therapy is the preferred treatment modality. Patients with metastatic prostate cancer eventually develop an androgen-refractory state within 12 to 18 months of treatment initiation, and approximately half of these patients die within 6 months thereafter. The most common site for prostate cancer metastasis is bone. Prostate cancer bone metastases are, on balance, characteristically osteoblastic rather than osteolytic (i.e., resulting in net bone formation). Bone metastases are found most frequently in the spine, followed by the femur, pelvis, rib cage, skull and humerus. Other common sites for metastasis include lymph nodes, lung, liver and brain. Metastatic prostate cancer is typically diagnosed by open or laparoscopic pelvic lymphadenectomy, whole body radionuclide scans, skeletal radiography, and/or bone lesion biopsy.

As used herein, the terms “locally advanced prostate cancer” and “locally advanced disease” mean prostate cancers which have extended through the prostate capsule, and are meant to include stage C disease under the American Urological Association (AUA) system, stage C1-C2 disease under the Whitmore-Jewett system, and stage T3-T4 and N+ disease under the TNM (tumor, node, metastasis) system. In general, surgery is not recommended for patients with locally advanced disease, and these patients have substantially less favorable outcomes compared to patients having clinically localized (organ-confined) prostate cancer. Locally advanced disease is clinically identified by palpable evidence of induration beyond the lateral border of the prostate or asymmetry or induration above the prostate base. Locally advanced prostate cancer is diagnosed pathologically following radical prostatectomy if the tumor invades or penetrates the prostatic capsule, extends into the surgical margin, or invades the seminal vesicles.

As used herein, “tumor stage” or “tumor progression” refers to the different clinical stages of the tumor. Clinical stages of a tumor are defined by various parameters which are well-established in the field of medicine. Some of the parameters include morphology, size of tumor, the degree in which it has metastasized through the patient's body and the like.

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

“Laser desorption mass spectrometer” refers to a mass spectrometer that uses laser energy as a means to desorb, volatilize, and ionize an analyte.

“Tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector—magnetic sector mass spectrometers, and combinations thereof.

“Mass analyzer” refers to a sub-assembly of a mass spectrometer that comprises means for measuring a parameter that can be translated into mass-to-charge ratios of gas phase ions. In a time-of-flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionucleides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. “Fluence” refers to the energy delivered per unit area of interrogated image. A high fluence source, such as a laser, will deliver about 1 mJ/mm2 to 50 mJ/mm2. Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons that ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Solid support” refers to a solid material which can be derivatized with, or otherwise attached to, a capture reagent. Exemplary solid supports include probes, microtiter plates and chromatographic resins.

“Probe” in the context of this invention refers to a device adapted to engage a probe interface of a gas phase ion spectrometer (e.g., a mass spectrometer) and to present an analyte to ionizing energy for ionization and introduction into a gas phase ion spectrometer, such as a mass spectrometer. A “probe” will generally comprise a solid substrate (either flexible or rigid) comprising a sample presenting surface on which an analyte is presented to the source of ionizing energy.

“Surface-enhanced laser desorption/ionization” or “SELDI” refers to a method of desorption/ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. SELDI technology is described in, e.g., U.S. Pat. No. 5,719,060 (Hutchens and Yip) and U.S. Pat. No. 6,225,047 (Hutchens and Yip).

“Surface-Enhanced Affinity Capture” or “SEAC” is a version of SELDI that involves the use of probes comprising an absorbent surface (a “SEAC probe”). “Adsorbent surface” refers to a surface to which is bound an adsorbent (also called a “capture reagent” or an “affinity reagent”). An adsorbent is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047 (Hutchens and Yip, “Use of retentate chromatography to generate difference maps,” May 1, 2001).

In some embodiments, a SEAC probe is provided as a pre-activated surface which can be modified to provide an adsorbent of choice. For example, certain probes are provided with a reactive moiety that is capable of binding a biological molecule through a covalent bond. Epoxide and carbodiimidizole are useful reactive moieties to covalently bind biospecific adsorbents such as antibodies or cellular receptors.

“Adsorption” refers to detectable non-covalent binding of an analyte to an adsorbent or capture reagent.

“Surface-Enhanced Neat Desorption” or “SEND” is a version of SELDI that involves the use of probes comprising energy absorbing molecules chemically bound to the probe surface. (“SEND probe.”) “Energy absorbing molecules” (“EAM”) refer to molecules that are capable of absorbing energy from a laser desorption/ ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. SEND is further described in U.S. Pat. No. 5,719,060 and U.S. patent application Ser. No. 60/408,255, filed Sep. 4, 2002 (Kitagawa, “Monomers And Polymers Having Energy Absorbing Moieties Of Use In Desorption/Ionization Of Analytes”).

“Surface-Enhanced Photolabile Attachment and Release” or “SEPAR” is a version of SELDI that involves the use of probes having moieties attached to the surface that can covalently bind an analyte, and then release the analyte through breaking a photolabile bond in the moiety after exposure to light, e.g., laser light. SEPAR is further described in U.S. Pat. No. 5,719,060.

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

“Analyte” refers to any component of a sample that is desired to be detected. The term can refer to a single component or a plurality of components in the sample.

The “complexity” of a sample adsorbed to an adsorption surface of an affinity capture probe means the number of different protein species that are adsorbed.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Monitoring” refers to recording changes in a continuously varying parameter.

“Biochip” refers to a solid substrate having a generally planar surface to which an adsorbent is attached. Frequently, the surface of the biochip comprises a plurality of addressable locations, each of which location has the adsorbent bound there. Biochips can be adapted to engage a probe interface and, therefore, function as probes.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO 00/56934 (Englert et al., “Continuous porous matrix arrays,” Sep. 28, 2000).

Protein biochips produced by Ciphergen Biosystems comprise surfaces having chromatographic or biospecific adsorbents attached thereto at addressable locations. Ciphergen ProteinChip® arrays include NP20, H4, H50, SAX-2, WCX-2, CM-10, IMAC-3, IMAC-30, LSAX-30, LWCX-30, IMAC-40, PS-10, PS-20 and PG-20. These protein biochips comprise an aluminum substrate in the form of a strip. The surface of the strip is coated with silicon dioxide.

In the case of the NP-20 biochip, silicon oxide functions as a hydrophilic adsorbent to capture hydrophilic proteins.

H4, H50, SAX-2, WCX-2, CM-10, IMAC-3, IMAC-30, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The H50 biochip has nonylphenoxy-poly(ethylene glycol)methacrylate for hydrophobic binding. The SAX-2 biochip has quaternary ammonium functionalities for anion exchange. The WCX-2 and CM-10 biochips have carboxylate functionalities for cation exchange. The IMAC-3 and IMAC-30 biochips have nitriloacetic acid functionalities that adsorb transition metal ions, such as Cu++ and Ni++, by chelation. These immobilized metal ions allow adsorption of peptide and proteins by coordinate bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The PG-20 biochip is a PS-20 chip to which Protein G is attached. The LSAX-30 (anion exchange), LWCX-30 (cation exchange) and IMAC-40 (metal chelate) biochips have functionalized latex beads on their surfaces. Such biochips are further described in: WO 00/66265 (Rich et al., “Probes for a Gas Phase Ion Spectrometer,” Nov. 9, 2000); WO 00/67293 (Beecher et al., “Sample Holder with Hydrophobic Coating for Gas Phase Mass Spectrometer,” Nov. 9, 2000); U.S. patent application Ser. No. US20030032043A1 (Pohl and Papanu, “Latex Based Adsorbent Chip,” Jul. 16, 2002) and U.S. patent application Ser. No. 60/350,110 (Um et al., “Hydrophobic Surface Chip,” Nov. 8, 2001).

Upon capture on a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

“Marker” in the context of the present invention refers to a polypeptide (of a particular apparent molecular weight), which is differentially present in a sample taken from patients having human cancer as compared to a comparable sample taken from control subjects (e.g., a person with a negative diagnosis or undetectable cancer, normal or healthy subject). The term “biomarker” is used interchangeably with the term “marker.”

The term “measuring” means methods which include detecting the presence or absence of marker(s) in the sample, quantifying the amount of marker(s) in the sample, and/or qualifying the type of biomarker. Measuring can be accomplished by methods known in the art and those further described herein, including but not limited to SELDI and immunoassay. Any suitable methods can be used to detect and measure one or more of the markers described herein. These methods include, without limitation, mass spectrometry (e.g., laser desorption/ionization mass spectrometry), fluorescence (e.g. sandwich immunoassay), surface plasmon resonance, ellipsometry and atomic force microscopy.

“Detect” refers to identifying the presence, absence or amount of the object to be detected.

The phrase “differentially present” refers to differences in the quantity and/or the frequency of a marker present in a sample taken from patients having human cancer as compared to a control subject. For example, some markers described herein are present at an elevated level in samples of cancer patients compared to samples from control subjects. In contrast, other markers described herein are present at a decreased level in samples of cancer patients compared to samples from control subjects. Furthermore, a marker can be a polypeptide, which is detected at a higher frequency or at a lower frequency in samples of human cancer patients compared to samples of control subjects. A marker can be differentially present in terms of quantity, frequency or both.

A polypeptide is differentially present between two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between two sets of samples if the frequency of detecting the polypeptide in the prostate cancer patients' samples is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition, i.e., prostate cancer. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of prostate cancer. A diagnostic amount can be either in absolute amount (e.g., μg/ml) or a relative amount (e.g., relative intensity of signals).

A “control amount” of a marker can be any amount or a range of amount, which is to be compared against a test amount of a marker. For example, a control amount of a marker can be the amount of a marker in a person without prostate cancer. A control amount can be either in absolute amount (e.g, μg/ml) or a relative amount (e.g., relative intensity of signals).

As used herein, the term “sensitivity” is the percentage of patients with a particular disease. For example, in the PCa/HC group, the biomarkers of the invention have a sensitivity of about 98%. The panel of biomarkers correctly classified 101 out of 103 prostate cancer patients as having prostate cancer, i.e. 101/103=98%.

As used herein, the term “specificity” is the percentage of patients correctly identified as having a particular disease i.e. normal or healthy subjects. For example, the specificity is calculated as the number of subjects with a particular disease as compared to normal healthy subjects.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

“Immunoassay” is an assay that uses an antibody to specifically bind an antigen (e.g., a marker). The immunoassay is characterized by the use of specific binding properties of a particular antibody to isolate, target, and/or quantify the antigen.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well-characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to marker “X” from specific species such as rat, mouse, or human can be selected to obtain only those polyclonal antibodies that are specifically immunoreactive with marker “X” and not with other proteins, except for polymorphic variants and alleles of marker “X”. This selection may be achieved by subtracting out antibodies that cross-react with marker “X” molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Managing subject treatment” refers to the behavior of the clinician or physician subsequent to the determination of prostate cancer status. For example, if the result of the methods of the present invention is inconclusive or there is reason that confirmation of status is necessary, the physician may order more tests. Alternatively, if the status indicates that surgery is appropriate, the physician may schedule the patient for surgery. Likewise, if the status is negative, e.g., late stage prostate cancer or if the status is acute, no further action may be warranted. Furthermore, if the results show that treatment has been successful, no further management may be necessary.

### DETAILED DESCRIPTION OF THE INVENTION

The present invention relates to a method for identification of tumor biomarkers markers for prostate cancer, with high specificity and sensitivity. In particular, a panel of biomarkers were identified that are associated with prostate cancer disease status. Using high throughput proteomic profiling and bioinformatics tools, twenty (20) biomarkers were identified that are associated with prostate cancer disease status based on results from serum samples collected from patients with: prostate cancer before treatment at various stages (n=103); men with benign prostate disease (n=60); and healthy men (n=19); without known prostate cancer. The corresponding proteins or fragments of proteins for the 20 biomarkers are represented as intensity peaks in SELDI (surface enhanced laser desorption/ionization) protein chip/mass spectra with molecular masses centered around 7808, 14576, 2062, 7973, 6677, and 3936 (Panel 1); 60958, 5149, 5861, 28098, 2996, 24346, 6722, 5999, 6159 and 55785 (Panel 2); and 2540, 8019, 4658 and 14703 (Panel 3), for the differentiation of prostate cancer (PCa) from healthy men as control (HC), PCa from benign prostatic hyperplasia (BPH), and non-organ confined PCa (pT3-4) from organ confined PCa (pT2), respectively. Using the three (3) panels of biomarkers, correctly differentiated the between the following diagnostic groups (% clinical sensitivity/specificity): PCa from HC=92%/90% using Panel 1, PCa from BPH=80%/77% using Panel 2, pT3-4 from pT2=90%/95% using Panel 3.

In particular, the panel of biomarkers: (Panel 3) with 90% sensitivity and 95% specificity can be used to select prostate cancer patients for surgery; Panel 1 with 92% sensitivity and 90% specificity can be used for the screening of prostate cancer in a healthy population of men and Panel 2 with 80% sensitivity and 77% specificity can be used for the diagnosis of prostate cancer and differentiate cancer from BPH.

### I. DESCRIPTION OF THE BIOMARKERS

The corresponding proteins or fragments of proteins for these biomarkers are represented as intensity peaks in SELDI (surface enhanced laser desorption/ionization) protein chip/mass spectra with molecular masses centered around the values indicated as follows.

Panel 1 biomarkers include the biomarkers identified as:
Marker I: having a molecular weight of about 7.808 kD
Marker II: having a molecular weight of about 14.576 kD
Marker III: having a molecular weight of about 2.062 kD
Marker IV: having a molecular weight of about 7.973 kD
Marker V: having a molecular weight of about 6.677 kD
Marker VI: having a molecular weight of about 3.936 kD.

Panel 2 biomarkers include the biomarkers identified as:
Marker VII: having a molecular weight of about 60.958 kD
Marker VIII: having a molecular weight of about 5.149 kD
Marker IX: having a molecular weight of about 5.861 kD
Marker X: having a molecular weight of about 28.098 kD
Marker XI: having a molecular weight of about 2.996 kD
Marker XII: having a molecular weight of about 24.346 kD
Marker XIII: having a molecular weight of about 6.722 kD
Marker XIV: having a molecular weight of about 5.999 kD
Marker XV: having a molecular weight of about 6.159 kD
Marker XVI: having a molecular weight of about 55.784 kD.

Additional Panel 2 biomarkers include the biomarkers identified as:
Marker XXI: having a molecular weight of about 2.68 kD
Marker XXII: having a molecular weight of about 3.16 kD
Marker XXIII: having a molecular weight of about 10.3 kD
Marker XXIV: having a molecular weight of about 10.8 kD
Marker XXV: having a molecular weight of about 12.7 kD, and
Marker XXVI: having a molecular weight of about 17.9 kD.

Panel 3 biomarkers include the biomarkers identified as:
Marker XVII: having a molecular weight of about 2.540 kD
Marker XVIII: having a molecular weight of about 8.019 kD
Marker XIX: having a molecular weight of about 4.658 kD, and
Marker XX: having a molecular weight of about 14.703 kD.

Additional Panel 3 biomarkers include the biomarkers identified as:
Marker XXVII: having a molecular weight of about 2.79 kD
Marker XXVIII: having a molecular weight of about 3.32 kD
Marker XXIX: having a molecular weight of about 4.29 kD
Marker XXX: having a molecular weight of about 15.9 kD
Marker XXXI: having a molecular weight of about 16.1 kD, and
Marker XXXII: having a molecular weight of about 16.3 kD.

These masses for Markers I through XXXII are considered accurate to within 0.15 percent of the specified value as determined by the disclosed SELDI-mass spectroscopy protocol.

As discussed above, Markers I through XXXII also may be characterized based on affinity for an adsorbent, particularly binding to an immobilized chelate (IMAC)-Nickel substrate surface under the conditions specified in the Examples, which follow.

The above-identified biomarkers, are examples of biomarkers, as determined by molecular weights, identified by the methods of the invention and serve merely as an illustrative example and are not meant to limit the invention in any way.

A major advantage of identification of these markers is their high specificity and ability to differentiate between different prostate disease states. This is of major importance when typical assays which measure PSA levels of less than 4 ng/ml. Not only is the presence of these markers indicative of prostate cancer but can differentiate between for example, Prostate cancer and benign prostatic hyperplasia (BPH), and non-organ confined prostate cancer from organ confined prostate cancer. Most prostate cancer patients have no known risk factors for tumor development or rate of disease progression. The markers of the invention are therefore important in monitoring and diagnosing for prostate cancer progression and to identify patients who are at risk for aggressive disease and would benefit from early treatment.

More specifically, the present invention is based upon the discovery of protein markers that are differentially present in samples of human cancer patients and control subjects, and the application of this discovery in methods and kits for aiding a human cancer diagnosis. Some of these protein markers are found at an elevated level and/or more frequently in samples from human cancer patients compared to a control (e.g., men in whom human cancer is undetectable). Accordingly, the amount of one or more markers found in a test sample compared to a control, or the mere detection of one or more markers in the test sample provides useful information regarding probability of whether a subject being tested has human cancer or not.

The protein markers of the present invention have a number of other uses. For example, the markers can be used to screen for compounds that modulate the expression of the markers in vitro or in vivo, which compounds in turn may be useful in treating or preventing human cancer in patients. In another example, markers can be used to monitor responses to certain treatments of human cancer. In yet another example, the markers can be used in the heredity studies. For instance, certain markers may be genetically linked. This can be determined by, e.g., analyzing samples from a population of human cancer patients whose families have a history of human cancer. The results can then be compared with data obtained from, e.g., human cancer patients whose families do not have a history of human cancer. The markers that are genetically linked may be used as a tool to determine if a subject whose family has a history of human cancer is pre-disposed to having human cancer.

In another aspect, the invention provides methods for detecting markers which are differentially present in the samples of a human cancer patient and a control (e.g., men in whom human cancer is undetectable). The markers can be detected in a number of biological samples. The sample is preferably a biological fluid sample. Examples of a biological fluid sample useful in this invention include blood, blood serum, plasma, nipple aspirate, urine, tears, saliva, etc. Because all of the markers are found in blood serum, blood serum is a preferred sample source for embodiments of the invention.

In accordance with the present invention, the methods described herein, pre-invasive or even benign tumors may be diagnosed by identifying the biomarkers which cause a pre-invasive tumor to progress to a malignant tumor. The type of biomarkers identified and amounts of biomarker may correlate with the jump from a pre-invasive tumor to a malignant stage tumor. Therapy such as immediate excision of the tumor or therapies such as chemotherapy or radiation therapy can be implemented prior to the tumor becoming invasive. The identification of the pre-invasive biomarkers can be used in diagnosis with conventional methods such as, for example, in prostate cancer, use of a digital rectal examination.

The biomarkers of the invention are highly specific in detecting and differentiating between diseases of the prostate such as, for example, prostate cancer at any stage; BPH, and the like. This is especially important when PSA levels are not determinative or cannot be detected by conventional or current means. For example, considerable uncertainty nevertheless remains about how to proceed with a patient who is PSA positive and DRE negative. In such cases the biomarkers of the invention are highly important in diagnosing the disease or non disease state of the prostate.

Current means of detecting diseases of the prostate are not determinative when a modestly abnormal PSA value (4-10 ng/ml) is encountered in the context of a negative digital rectal exam (DRE). In such cases, only 20-30% of individuals with such findings will demonstrate carcinoma on biopsy (Kantoff and Talcott, 8(3) Hematol. Oncol. Clinics N Amer 555 (1994)). Thus, in accordance with the invention detection of at least one biomarker is desirable in differentiating between benign, pre-invasive, malignant and different stages of prostate cancer. An illustrative example of biomarkers which differentiate between the different prostate cancer stages or normal prostate are shown in Tables 1 through 3.

In addition, PSA is not a disease-specific marker, as elevated levels of PSA are detectable in a large percentage of patients with BPH and prostatitis (25-86%) (Gao et al., 1997, Prostate 31: 264-281), as well as in other nonmalignant disorders and in some normal men, a factor which significantly limits the diagnostic specificity of this marker. For example, elevations in serum PSA of between 4 to 10 ng/ml are observed in BPH, and even higher values are observed in prostatitis, particularly acute prostatitis. BPH is an extremely common condition in men. Further confusing the situation is the fact that serum PSA elevations may be observed without any indication of disease from DRE, and vice-versa. Moreover, it is now recognized that PSA is not prostate-specific (Gao et al., supra, for review). The problems with current methodologies underscore the importance of detection of the biomarkers of the invention. Thus, in accordance with the invention, detection of at least one biomarker is preferred in differentiating between the different diseases states of the prostate. An illustrative example of biomarkers which differentiate between the different disease state or normal prostate are shown in Tables 1 through 3.

Any suitable methods can be used to detect one or more of the markers described herein. These methods include, without limitation, mass spectrometry (e.g., laser desorption/ionization mass spectrometry), fluorescence (e.g sandwich immunoassay), surface plasmon resonance, ellipsometry and atomic force microscopy.

The following example is illustrative of the methods used to identify biomarkers for detection of prostate diseases. It is not meant to limit or construe the invention in any way. A sample, such as for example, serum from a subject or patient, is immobilized on a biochip. Preferably, the biochip comprises a functionalized, cross-linked polymer in the form of a hydrogel physically attached to the surface of the biochip or covalently attached through a silane to the surface of the biochip. However, any biochip which can bind samples from subjects can be used. The surfaces of the biochips are comprised of, for example, hydrophilic adsorbent to capture hydrophilic proteins (e.g. silicon oxide); carboimidizole functional groups that can react with groups on proteins for covalent binding; epoxide functional groups for covalent binding with proteins (e.g. antibodies, receptors, lectins, heparin, Protein A, biotin/streptavidin and the like); anionic exchange groups; cation exchange groups; metal chelators and the like.

Preferably, samples are pre-fractionated prior to immobilization as discussed below. Analytes or samples captured on the surface of a biochip can be detected by any method known in the art. This includes, for example, mass spectrometry, fluorescence, surface plasmon resonance, ellipsometry and atomic force microscopy. Mass spectrometry, and particularly SELDI mass spectrometry, is a particularly useful method for detection of the biomarkers of this invention.

Immobilized samples or analytes are preferably subjected to laser ionization and the intensity of signal for mass/charge ratio is detected. The data obtained from the mass/charge ratio signal is transformed into data which is read by any type of computer. An algorithm is executed by the computer user that classifies the data according to user input parameters, for detecting signals that represent biomarkers present in, for example, prostate cancer patients and are lacking in non-cancer subject controls. The biomarkers are most preferably identified by their molecular weights.

### II. TEST SAMPLES

### A) Subject Types

Samples are collected from subjects, e.g., men, who want to establish prostate cancer status. The subjects may be men who have been determined to have a high risk of prostate cancer based on their family history. Other patients include men who have prostate cancer and the test is being used to determine the effectiveness of therapy or treatment they are receiving. Also, patients could include healthy men who are having a test as part of a routine examination, or to establish baseline levels of the biomarkers. Samples may be collected from men who had been diagnosed with prostate cancer and received treatment to eliminate the cancer, or perhaps are in remission.

### B) Types of Sample and Preparation of the Sample

The markers can be measured in different types of biological sanples. The sample is preferably a biological fluid sample. Examples of a biological fluid sample useful in this invention include blood, blood plasma, serum, urine, tissue, cells, organs and seminal fluids, etc. Because all of the markers are found in blood serum, blood serum is a preferred sample source for embodiments of the invention.

If desired, the sample can be prepared to enhance detectability of the markers. Typically, preparation involves fractionation of the sample and collection of fractions determined to contain the biomarkers. Methods of pre-fractionation include, for example, size exclusion chromatography, ion exchange chromatography, heparin chromatography, affinity chromatography, sequential extraction, gel electrophoresis and liquid chromatography. The analytes also may be modified prior to detection. These methods are useful to simplify the sample for further analysis. For example, it can be useful to remove high abundance proteins, such as albumin, from blood before analysis.

In one embodiment, a sample can be pre-fractionated according to size of proteins in a sample using size exclusion chromatography. For a biological sample wherein the amount of sample available is small, preferably a size selection spin column is used. For example, a K30 spin column (available from Princeton Separation, Ciphergen Biosystems, Inc., etc.) can be used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by gas phase ion spectrometry for the detection of markers.

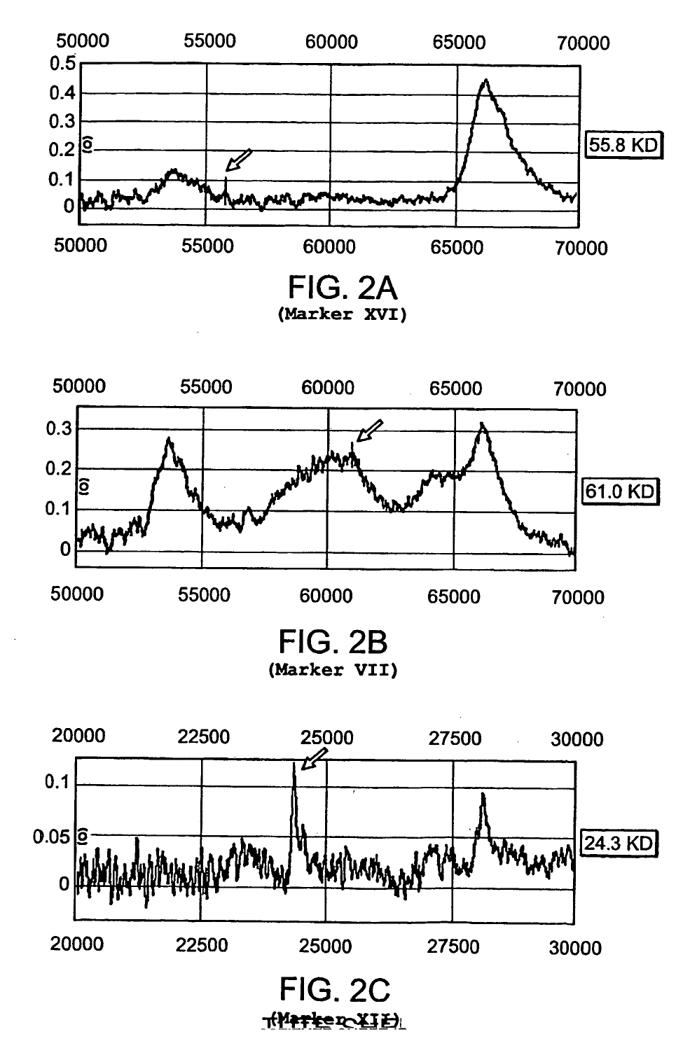

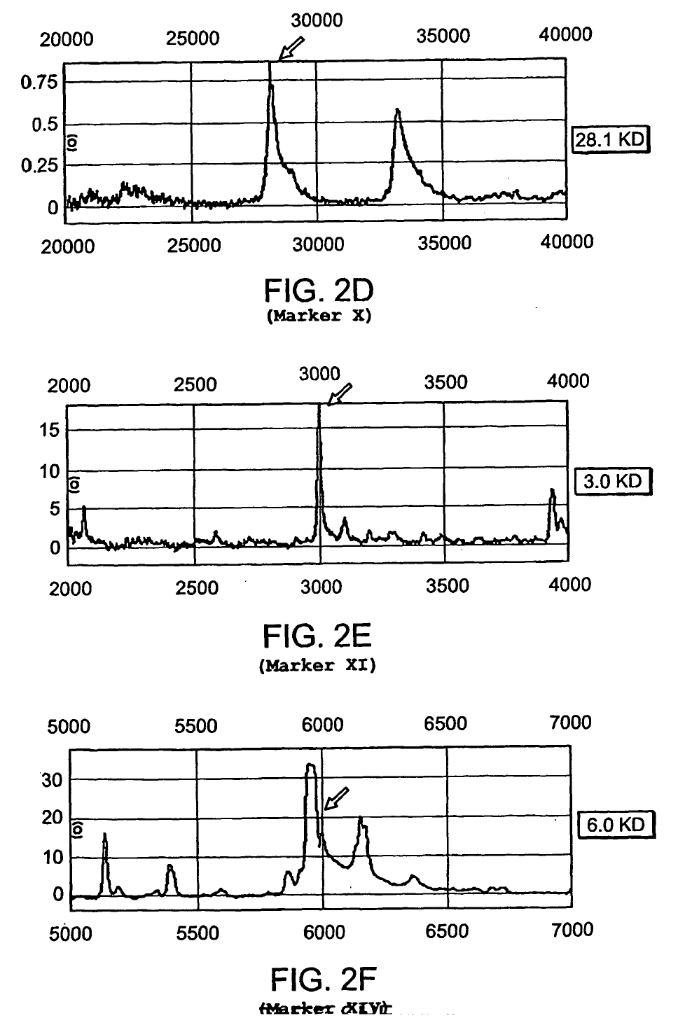

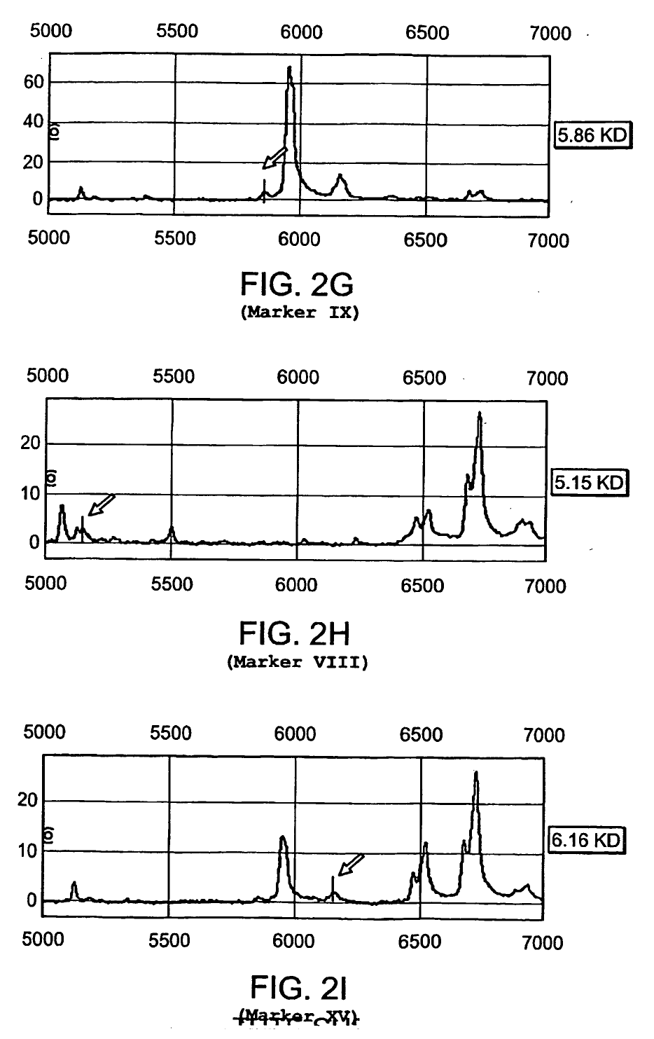

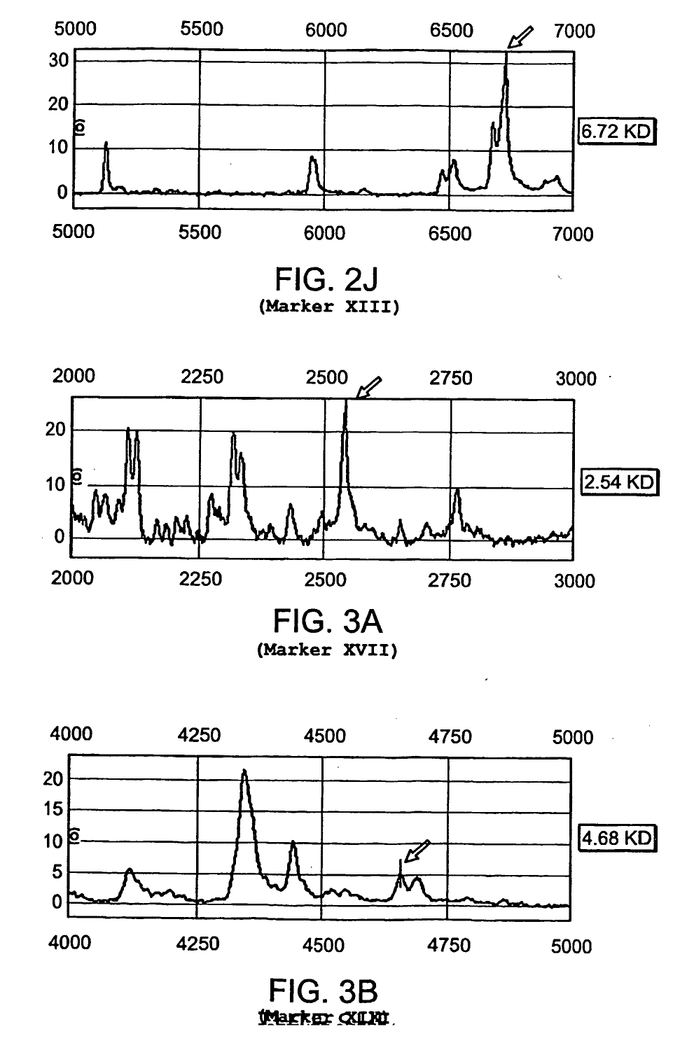

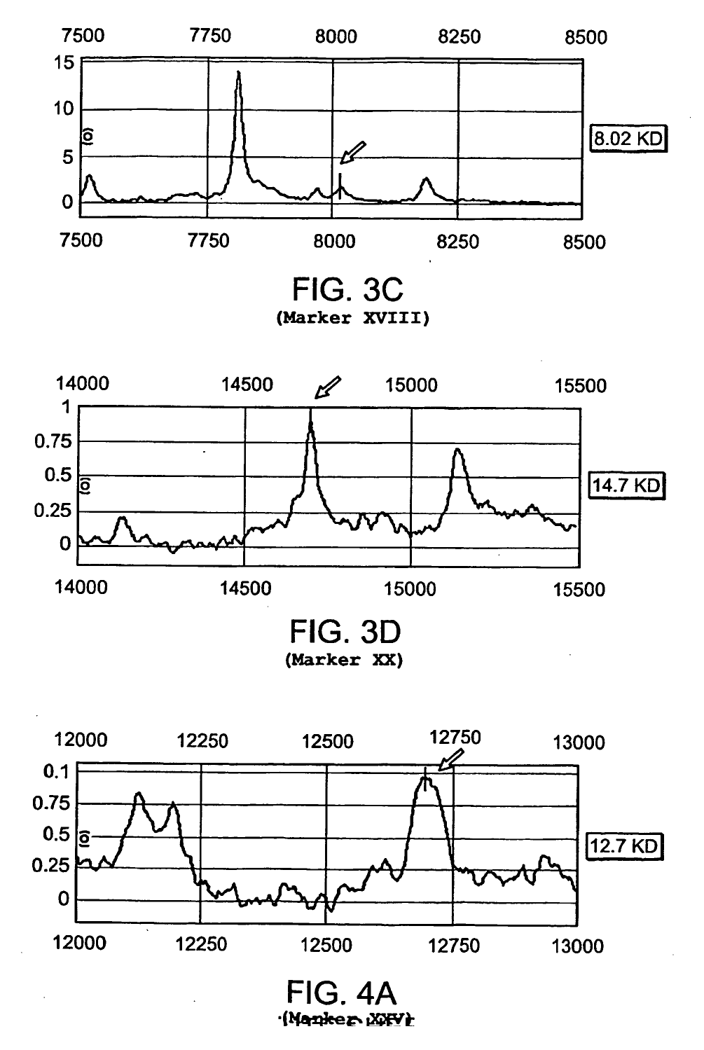

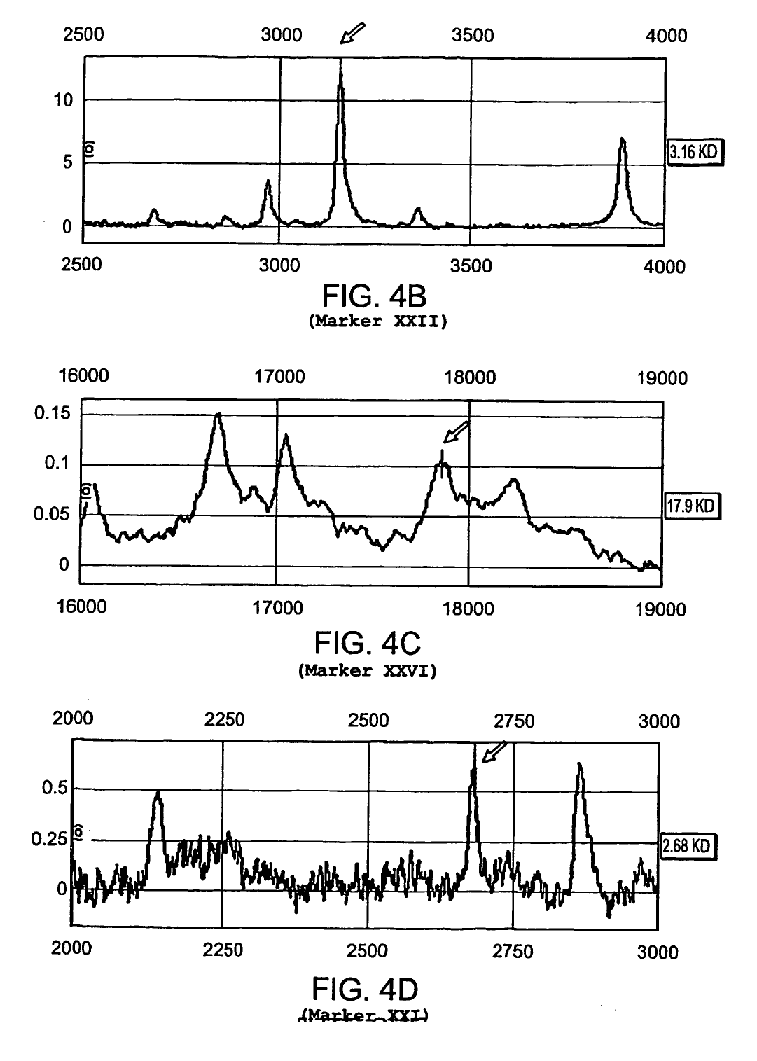

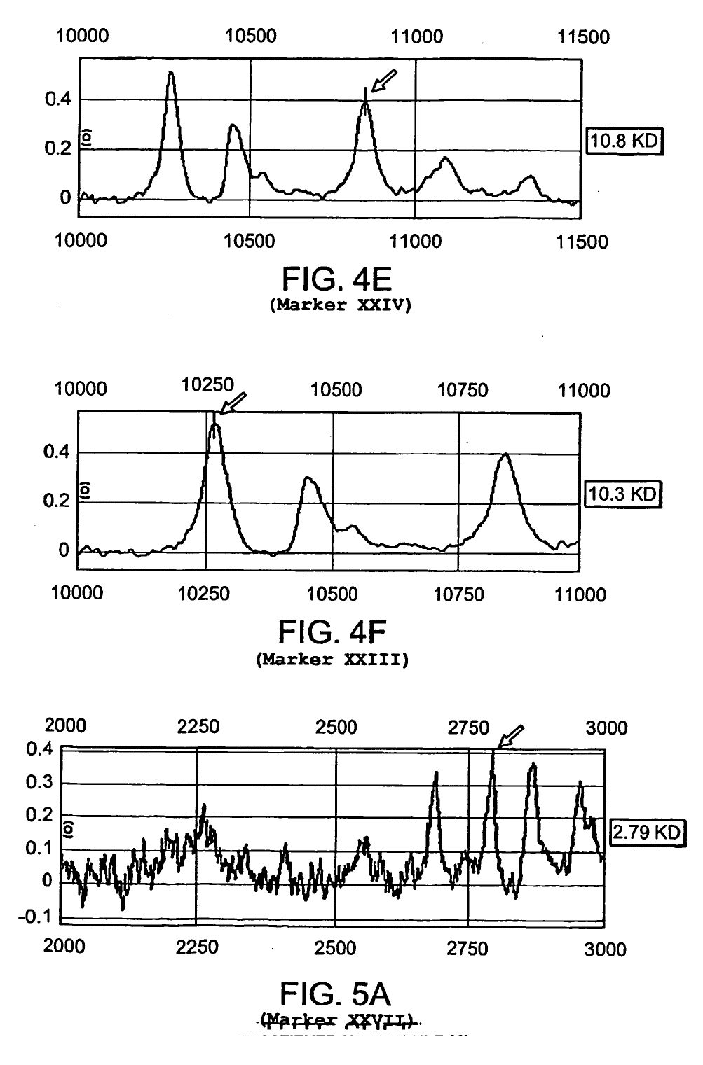

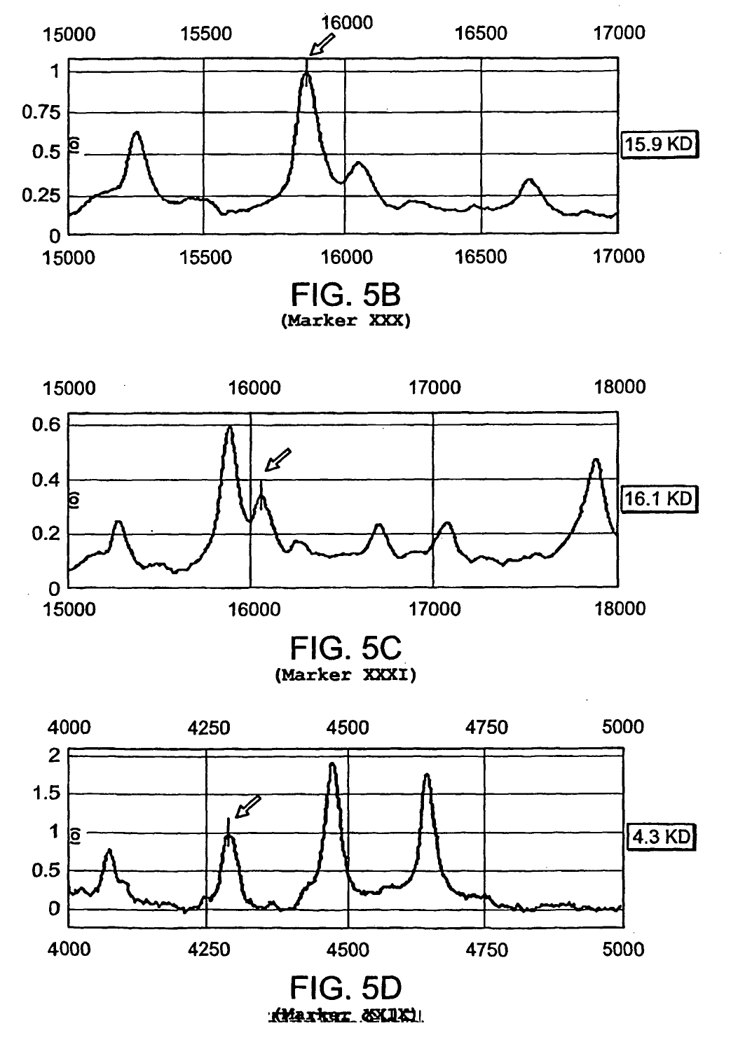

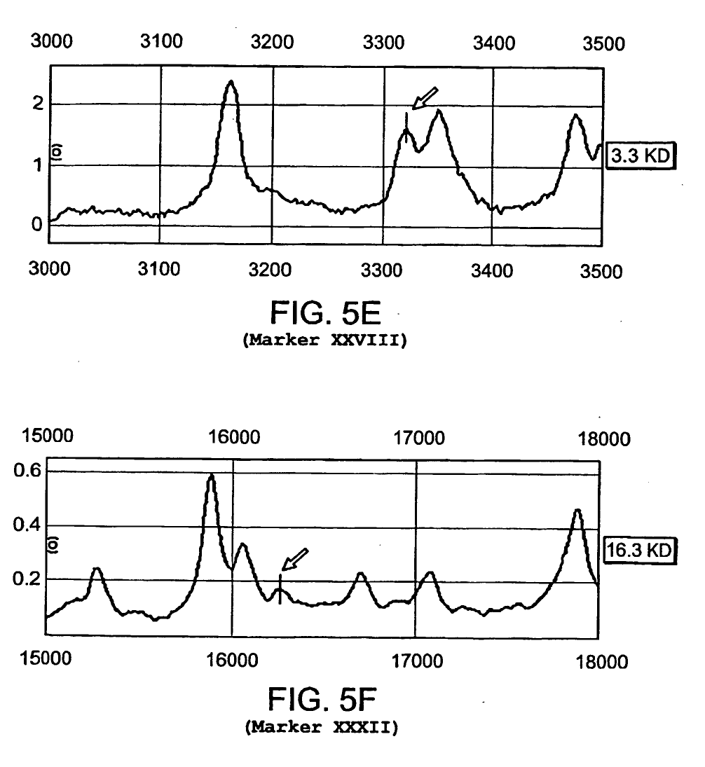

In another embodiment, a sample can be pre-fractionated by anion exchange chromatography. Anion exchange chromatography allows pre-fractionation of the proteins in a sample roughly according to their charge characteristics. For example, a Q anion-exchange resin can be used (e.g., Q HyperD F, Biosepra), and a sample can be sequentially eluted with eluants having different pH's (see FIG. 2 and Example section VIB). Anion exchange chromatography allows separation of biomolecules in a sample that are more negatively charged from other types of biomolecules. Proteins that are eluted with an eluant having a high pH is likely to be weakly negatively charged, and a fraction that is eluted with an eluant having a low pH is likely to be strongly negatively charged. Thus, in addition to reducing complexity of a sample, anion exchange chromatography separates proteins according to their binding characteristics.

In yet another embodiment, a sample can be pre-fractionated by heparin chromatography. Heparin chromatography allows pre-fractionation of the markers in a sample also on the basis of affinity interaction with heparin and charge characteristics. Heparin, a sulfated mucopolysaccharide, will bind markers with positively charged moieties and a sample can be sequentially eluted with eluants having different pH's or salt concentrations. Markers eluted with an eluant having a low pH are more likely to be weakly positively charged. Markers eluted with an eluant having a high pH are more likely to be strongly positively charged. Thus, heparin chromatography also reduces the complexity of a sample and separates markers according to their binding characteristics.

In yet another embodiment, a sample can be pre-fractionated by removing proteins that are present in a high quantity or that may interfere with the detection of markers in a sample. For example, in a blood serum sample, serum albumin is present in a high quantity and may obscure the analysis of markers. Thus, a blood serum sample can be pre-fractionated by removing serum albumin. Serum albumin can be removed using a substrate that comprises adsorbents that specifically bind serum albumin. For example, a column which comprises, e.g., Cibacron blue agarose (which has a high affinity for serum albumin) or anti-serum albumin antibodies can be used.

In yet another embodiment, a sample can be pre-fractionated by isolating proteins that have a specific characteristic, e.g. are glycosylated. For example, a blood serum sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

Many types of affinity adsorbents exist which are suitable for pre-fractionating blood serum samples. An example of one other type of affinity chromatography available to pre-fractionate a sample is a single stranded DNA spin column. These columns bind proteins which are basic or positively charged. Bound proteins are then eluted from the column using eluants containing denaturants or high pH.

Thus there are many ways to reduce the complexity of a sample based on the binding properties of the proteins in the sample, or the characteristics of the proteins in the sample.

In yet another embodiment, a sample can be fractionated using a sequential extraction protocol. In sequential extraction, a sample is exposed to a series of adsorbents to extract different types of biomolecules from a sample. For example, a sample is applied to a first adsorbent to extract certain proteins, and an eluant containing non-adsorbent proteins (i.e., proteins that did not bind to the first adsorbent) is collected. Then, the fraction is exposed to a second adsorbent. This further extracts various proteins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomolecules. In this embodiment, the sample is applied to a first adsorbent on the probe, which is subsequently washed with an eluant. Markers that do not bind to the first adsorbent is removed with an eluant. The markers that are in the fraction can be applied to a second adsorbent on the probe, and so forth. The advantage of performing sequential extraction on a gas phase ion spectrometer probe is that markers that bind to various adsorbents at every stage of the sequential extraction protocol can be analyzed directly using a gas phase ion spectrometer.

In yet another embodiment, biomolecules in a sample can be separated by high-resolution electrophoresis, e.g., one or two-dimensional gel electrophoresis. A fraction containing a marker can be isolated and further analyzed by gas phase ion spectrometry. Preferably, two-dimensional gel electrophoresis is used to generate two-dimensional array of spots of biomolecules, including one or more markers. See, e.g., Jungblut and Thiede, Mass Spectr. Rev. 16:145-162 (1997).

The two-dimensional gel electrophoresis can be performed using methods known in the art. See, e.g., Deutscher ed., Methods In Enzymology vol. 182. Typically, biomolecules in a sample are separated by, e.g., isoelectric focusing, during which biomolecules in a sample are separated in a pH gradient until they reach a spot where their net charge is zero (i.e., isoelectric point). This first separation step results in one-dimensional array of biomolecules. The biomolecules in one dimensional array is further separated using a technique generally distinct from that used in the first separation step. For example, in the second dimension, biomolecules separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of biomolecules. Typically, two-dimensional gel electrophoresis can separate chemically different biomolecules in the molecular mass range from 1000-200,000 Da within complex mixtures.

Biomolecules in the two-dimensional array can be detected using any suitable methods known in the art. For example, biomolecules in a gel can be labeled or stained (e.g., Coomassie Blue or silver staining). If gel electrophoresis generates spots that correspond to the molecular weight of one or more markers of the invention, the spot can be is further analyzed by gas phase ion spectrometry. For example, spots can be excised from the gel and analyzed by gas phase ion spectrometry. Alternatively, the gel containing biomolecules can be transferred to an inert membrane by applying an electric field. Then a spot on the membrane that approximately corresponds to the molecular weight of a marker can be analyzed by gas phase ion spectrometry. In gas phase ion spectrometry, the spots can be analyzed using any suitable techniques, such as MALDI or SELDI (e.g., using ProteinChip® array) as described in detail below.

Prior to gas phase ion spectrometry analysis, it may be desirable to cleave biomolecules in the spot into smaller fragments using cleaving reagents, such as proteases (e.g., trypsin). The digestion of biomolecules into small fragments provides a mass fingerprint of the biomolecules in the spot, which can be used to determine the identity of markers if desired.

In yet another embodiment, high performance liquid chromatography (HPLC) can be used to separate a mixture of biomolecules in a sample based on their different physical properties, such as polarity, charge and size. HPLC instruments typically consist of a reservoir of mobile phase, a pump, an injector, a separation column, and a detector. Biomolecules in a sample are separated by injecting an aliquot of the sample onto the column. Different biomolecules in the mixture pass through the column at different rates due to differences in their partitioning behavior between the mobile liquid phase and the stationary phase. A fraction that corresponds to the molecular weight and/or physical properties of one or more markers can be collected. The fraction can then be analyzed by gas phase ion spectrometry to detect markers. For example, the spots can be analyzed using either MALDI or SELDI (e.g., using ProteinChip® array) as described in detail below.

Optionally, a marker can be modified before analysis to improve its resolution or to determine its identity. For example, the markers may be subject to proteolytic digestion before analysis. Any protease can be used. Proteases, such as trypsin, that are likely to cleave the markers into a discrete number of fragments are particularly useful. The fragments that result from digestion function as a fingerprint for the markers, thereby enabling their detection indirectly. This is particularly useful where there are markers with similar molecular masses that might be confused for the marker in question. Also, proteolytic fragmentation is useful for high molecular weight markers because smaller markers are more easily resolved by mass spectrometry. In another example, biomolecules can be modified to improve detection resolution. For instance, neuraminidase can be used to remove terminal sialic acid residues from glycoproteins to improve binding to an anionic adsorbent (e.g., cationic exchange ProteinChip® arrays) and to improve detection resolution. In another example, the markers can be modified by the attachment of a tag of particular molecular weight that specifically bind to molecular markers, further distinguishing them. Optionally, after detecting such modified markers, the identity of the markers can be further determined by matching the physical and chemical characteristics of the modified markers in a protein database (e.g., SwissProt).

### III. CAPTURE OF MARKERS

Biomarkers are preferably captured with capture reagents immobilized to a solid support, such as any biochip described herein, a multiwell microtiter plate, a resin, or nitrocellulose membranes that are subsequently probed for the presence of proteins. In particular, the biomarkers of this invention are preferably captured on SELDI protein biochips. Capture can be on a chromatographic surface or a biospecific surface. Any of the SELDI protein biochips comprising reactive surfaces can be used to capture and detect the biomarkers of this invention. However, the biomarkers of this invention bind well to immobilized metal chelates. The IMAC-3 and IMAC 30 biochips, which nitriloacetic acid functionalities that adsorb transition metal ions, such as Cu⁺⁺ and Ni⁺⁺, by chelation, are the preferred SELDI biochips for capturing the biomarkers of this invention. Any of the SELDI protein biochips comprising reactive surfaces can be used to capture and detect the biomarkers of this invention. These biochips can be derivatized with the antibodies that specifically capture the biomarkers, or they can be derivatized with capture reagents, such as protein A or protein G that bind immunoglobulins. Then the biomarkers can be captured in solution using specific antibodies and the captured markers isolated on chip through the capture reagent.

In general, a sample containing the biomarkers, such as serum, is placed on the active surface of a biochip for a sufficient time to allow binding. Then, unbound molecules are washed from the surface using a suitable eluant, such as phosphate buffered saline. In general, the more stringent the eluant, the more tightly the proteins must be bound to be retained after the wash. The retained protein biomarkers now can be detected by appropriate means.

### IV. DETECTION AND MEASUREMENT OF MARKERS

Once captured on a substrate, e.g., biochip or antibody, any suitable method can be used to measure a marker or markers in a sample. For example, markers can be detected and/or measured by a variety of detection methods including for example, gas phase ion spectrometry methods, optical methods, electrochemical methods, atomic force microscopy, radio frequency methods, surface plasmon resonance, ellipsometry and atomic force microscopy.

### A) SELDI

One preferred method of detection and/or measurement of the biomarkers uses mass spectrometry and, in particular, “Surface-enhanced laser desorption/ionization” or “SELDI”. SELDI refers to a method of desorption/ionization gas phase ion spectrometry (e.g., mass spectrometry) in which the analyte is captured on the surface of a SELDI probe that engages the probe interface. In “SELDI MS,” the gas phase ion spectrometer is a mass spectrometer. SELDI technology is described in more detail above and as follows.

Preferably, a laser desorption time-of-flight mass spectrometer is used in embodiments of the invention. In laser desorption mass .spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Markers on the substrate surface can be desorbed and ionized using gas phase ion spectrometry. Any suitable gas phase ion spectrometers can be used as long as it allows markers on the substrate to be resolved. Preferably, gas phase ion spectrometers allow quantitation of markers.

In one embodiment, a gas phase ion spectrometer is a mass spectrometer. In a typical mass spectrometer, a substrate or a probe comprising markers on its surface is introduced into an inlet system of the mass spectrometer. The markers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of markers or other substances will typically involve detection of signal intensity. This, in turn, can reflect the quantity and character of markers bound to the substrate. Any of the components of a mass spectrometer (e.g., a desorption source, a mass analyzer, a detector, etc.) can be combined with other suitable components described herein or others known in the art in embodiments of the invention.

Preferably, a laser desorption time-of-flight mass spectrometer is used in embodiments of the invention. In laser desorption mass spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

In another embodiment, an ion mobility spectrometer can be used to detect markers. The principle of ion mobility spectrometry is based on different mobility of ions. Specifically, ions of a sample produced by ionization move at different rates, due to their difference in, e.g., mass, charge, or shape, through a tube under the influence of an electric field. The ions (typically in the form of a current) are registered at the detector which can then be used to identify a marker or other substances in a sample. One advantage of ion mobility spectrometry is that it can operate at atmospheric pressure.

In yet another embodiment, a total ion current measuring device can be used to detect and characterize markers. This device can be used when the substrate has a only a single type of marker. When a single type of marker is on the substrate, the total current generated from the ionized marker reflects the quantity and other characteristics of the marker. The total ion current produced by the marker can then be compared to a control (e.g., a total ion current of a known compound). The quantity or other characteristics of the marker can then be determined.

### B) IMMUNOASSAY

In another embodiment, an immunoassay can be used to detect and analyze markers in a sample. This method comprises: (a) providing an antibody that specifically binds to a marker; (b) contacting a sample with the antibody; and (c) detecting the presence of a complex of the antibody bound to the marker in the sample.

An immunoassay is an assay that uses an antibody to specifically bind an antigen (e.g., a marker). The immunoassay is characterized by the use of specific binding properties of a particular antibody to isolate, target, and/or quantify the antigen. The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a marker from specific species such as rat, mouse, or human can be selected to obtain only those polyclonal antibodies that are specifically inununoreactive with that marker and not with other proteins, except for polymorphic variants and alleles of the marker. This selection may be achieved by subtracting out antibodies that cross-react with the marker molecules from other species.

Using the purified markers or their nucleic acid sequences, antibodies that specifically bind to a marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Typically a specific or selective reaction will be at least twice background signal or noise and more typically more than 10 to 100 times background.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the fonn of, e.g., a microtiter plate, a stick, a bead, or a microbead. Antibodies can also be attached to a probe substrate or ProteinChip® array described above. The sample is preferably a biological fluid sample taken from a subject. Examples of biological fluid samples include blood, serum, plasma, nipple aspirate, urine, tears, saliva etc. In a preferred embodiment, the biological fluid comprises blood serum. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Methods for measuring the amount of, or presence of, antibody-marker complex include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Electrochemical methods include voltametry and amperometry methods. Radio frequency methods include multipolar resonance spectroscopy. Methods for performing these assays are readily known in the art. Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

Immunoassays can be used to determine presence or absence of a marker in a sample as well as the quantity of a marker in a sample. The amount of an antibody-marker complex can be determined by comparing to a standard. A standard can be, e.g., a known compound or another protein known to be present in a sample. As noted above, the test amount of marker need not be measured in absolute units, as long as the unit of measurement can be compared to a control.

The methods for detecting these markers in a sample have many applications. For example, one or more markers can be measured to aid human cancer diagnosis or prognosis. In another example, the methods for detection of the markers can be used to monitor responses in a subject to cancer treatment. In another example, the methods for detecting markers can be used to assay for and to identify compounds that modulate expression of these markers in vivo or in vitro. In a preferred example, the biomarkers are used to differentiate between the different stages of tumor progression, thus aiding in determining appropriate treatment and extent of metastasis of the tumor.

### V. DATA ANALYSIS

The methods for detecting these markers in a sample have many applications. For example, one or more markers can be measured to aid human cancer diagnosis or prognosis. In another example, the methods for detection of the markers can be used to monitor responses in a subject to cancer treatment. In another example, the methods for detecting markers can be used to assay for and to identify compounds that modulate expression of these markers in vivo or in vitro.

Data generated by desorption and detection of markers can be analyzed using any suitable means. In one embodiment, data is analyzed with the use of a programmable digital computer. The computer program generally contains a readable medium that stores codes. Certain code can be devoted to memory that includes the location of each feature on a probe, the identity of the adsorbent at that feature and the -elution conditions used to wash the adsorbent. The computer also contains code that receives as input, data on the strength of the signal at various molecular masses received from a particular addressable location on the probe. This data can indicate the number of markers detected, including the strength of the signal generated by each marker.

Data analysis can include the steps of determining signal strength (e.g., height of peaks) of a marker detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other biomolecules can be displayed in the form of relative intensities in the scale desired (e.g., 100). Alternatively, a standard (e.g., a serum protein) may be admitted with the sample so that a peak from the standard can be used as a reference to calculate relative intensities of the signals observed for each marker or other markers detected.

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique markers and markers which are up- or down-regulated between samples. Marker profiles (spectra) from any two samples may be compared visually. In yet another format, Spotfire Scatter Plot can be used, wherein markers that are detected are plotted as a dot in a plot, wherein one axis of the plot represents the apparent molecular of the markers detected and another axis represents the signal intensity of markers detected. For each sample, markers that are detected and the amount of markers present in the sample can be saved in a computer readable medium. This data can then be compared to a control (e.g., a profile or quantity of markers detected in control, e.g., men in whom human cancer is undetectable).

When the sample is measured and data is generated, e.g., by mass spectrometry, the data is then analyzed by a computer software program. Generally, the software can comprise code that converts signal from the mass spectrometer into computer readable form. The software also can include code that applies an algorithm to the analysis of the signal to determine whether the signal represents a “peak” in the signal corresponding to a marker of this invention, or other useful markers. The software also can include code that executes an algorithm that compares signal from a test sample to a typical signal characteristic of “normal” and human cancer and determines the closeness of fit between the two signals. The software also can include code indicating which the test sample is closest to, thereby providing a probable diagnosis.

In preferred methods of the present invention, multiple biomarkers are measured. The use of multiple biomarkers increases the predictive value of the test and provides greater utility in diagnosis, toxicology, patient stratification and patient monitoring. The process called “Pattern recognition” detects the patterns formed by multiple biomarkers greatly improves the sensitivity and specificity of clinical proteomics for predictive medicine. Subtle variations in data from clinical samples, e.g., obtained using SELDI, indicate that certain patterns of protein expression can predict phenotypes such as the presence or absence of a certain disease, a particular stage of cancer progression, or a positive or adverse response to drug treatments.

Data generation in mass spectrometry begins with the detection of ions by an ion detector as described above. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering.

TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

In one example, Ciphergen's Biomarker Patterns™ Software is used to detect a pattern in the spectra that are generated. The data is classified using a pattern recognition process that uses a classification model. In general, the spectra will represent samples from at least two different groups for which a classification algorithm is sought. For example, the groups can be pathological v. non-pathological (e.g., cancer v. non-cancer), drug responder v. drug non-responder, toxic response v. non-toxic response, progressor to disease state v. non-progressor to disease state, phenotypic condition present v. phenotypic condition absent.

The spectra that are generated in embodiments of the invention can be classified using a pattern recognition process that uses a classification model. In some embodiments, data derived from the spectra (e.g., mass spectra or time-of-flight spectra) that are generated using samples such as “known samples” can then be used to “train” a classification model. A “known sample” is a sample that is pre-classified (e.g., cancer or not cancer). Data derived from the spectra (e.g., mass spectra or time-of-flight spectra) that are generated using samples such as “known samples” can then be used to “train” a classification model. A “known sample” is a sample that is pre-classified. The data that are derived from the spectra and are used to form the classification model can be referred to as a “training data set”. Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased vs. non diseased).

The training data set that is used to form the classification model may comprise raw data or pre-processed data. In some embodiments, raw data can be obtained directly from time-of-flight spectra or mass spectra, and then may be optionally “pre-processed” in any suitable manner. For example, signals above a predetermined signal-to-noise ratio can be selected so that a subset of peaks in a spectrum is selected, rather than selecting all peaks in a spectrum. In another example, a predetermined number of peak “clusters” at a common value (e.g., a particular time-of-flight value or mass-to-charge ratio value) can be used to select peaks. Illustratively, if a peak at a given mass-to-charge ratio is in less than 50% of the mass spectra in a group of mass spectra, then the peak at that mass-to-charge ratio can be omitted from the training data set. Pre-processing steps such as these can be used to reduce the amount of data that is used to train the classification model.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, which is herein incorporated by reference in its entirety.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART - classification and regression trees), artificial neural networks such as backpropagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. 2002 0138208 A1 (Paulse et al., “Method for analyzing mass spectra,” Sep. 26, 2002.

In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described in, for example, WO 01/31580 (Barnhill et al., “Methods and devices for identifying patterns in biological systems and methods of use thereof,” May 3, 2001); U.S. 2002/0193950 Al (Gavin et al., “Method or analyzing mass spectra,” Dec. 19, 2002); U.S. 2003/0004402 A1 (Hitt et al., “Process for discriminating between biological states based on hidden patterns from biological data,” Jan. 2, 2003); and U.S. 2003/0055615 A1 (Zhang and Zhang, “Systems and methods for processing biological expression data” Mar. 20, 2003).

More specifically, to obtain the biomarkers the peak intensity data of samples from cancer patients and healthy controls are used as a “discovery set.” This data were combined and randomly divided into a training set and a test set to construct and test multivariate predictive models using a non-linear version of Unified Maximum Separability Analysis (“USMA”) classifiers. Details of USMA classifiers are described in U.S. 2003/0055615 A1.

Generally, the data generated from Section IV above is inputted into a diagnostic algorithm (i.e., classification algorithm as described above). The classification algorithm is then generated based on the learning algorithm. The process involves developing an algorithm that can generate the classification algorithm. The methods of the present invention generate a more accurate classification algorithm by accessing a number of prostate cancer and normal samples of a sufficient number based on statistical sample calculations. The samples are used as a training set of data on learning algorithm.

The generation of the classification, i.e., diagnostic, algorithm is dependent upon the assay protocol used to analyze samples and generate the data obtained in Section IV above. It is imperative that the protocol for the detection and/or measurement of the markers (e.g., in step IV) must be the same as that used to obtain the data used for developing the classification algorithm. The assay conditions, which must be maintained throughout the training and classification systems include chip type and mass spectrometer parameters, as well as general protocols for sample preparation and testing. If the protocol for the detection and/or measurement of the markers (step IV) is changed, the learning algorithm and classification algorithm must also change. Similarly, if the learning algorithm and classification algorithm change, then the protocol for the detection and/or measurement of markers (step IV) must also change to be consistent with that used to generate classification algorithm. Development of a new classification model would require accessing a sufficient number of prostate cancer and normal samples, developing a new training set of data based on a new detection protocol, generating a new classification algorithm using the data and finally, verifying the classification algorithm with a multi-site study.

The classification models can be formed on and used on any suitable digital computer. Suitable digital computers include micro, mini, or large computers using any standard or specialized operating system such as a Unix, Windows™ or Linux™ based operating system. The digital computer that is used may be physically separate from the mass spectrometer that is used to create the spectra of interest, or it may be coupled to the mass spectrometer. If it is separate from the mass spectrometer, the data must be inputted into the computer by some other means, whether manually or automated.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

### VI. EXAMPLES OF PREFERRED EMBODIMENTS

The invention provides methods for aiding a human cancer diagnosis using one or more markers, for example Markers in panels 1, 2 and 3. These markers can be used alone, in combination with other markers in any set, or with entirely different markers (e.g., PSA antigen) in aiding human cancer diagnosis. The markers are differentially present in samples of a human cancer patient, for example prostate cancer patient, and a normal subject in whom human cancer is undetectable. For example, some of the markers are expressed at an elevated level and/or are present at a higher frequency in human cancer patients than in normal subjects. Therefore, detection of one or more of these markers in a person would provide useful information regarding the probability that the person may have human cancer.

In a preferred embodiment, a serum sample is collected from a patient and then fractionated using an anion exchange resin as described above. The biomarkers in the sample are captured using an IMAC3 nickel ProteinChip array. The markers are then detected using SELDI. The results are then entered into a computer system, which contains an algorithm that is designed using the same parameters that were used in the learning algorithm and classification algorithm to originally determine the biomarkers. The algorithm produces a diagnosis based upon the data received relating to each biomarker.

The diagnosis is determined by examining the data produced from the SELDI tests with the classification algorithm that is developed using the biomarkers. The classification algorithm depends on the particulars of the test protocol used to detect the biomarkers. These particulars include, for example, sample preparation, chip type and mass spectrometer parameters. If the test parameters change, the algorithm must change. Similarly, if the algorithm changes, the test protocol must change.

In another embodiment, the sample is collected from the patient. The biomarkers are captured using an antibody ProteinChip array as described above. The markers are detected using a biospecific SELDI test system. The results are then entered into a computer system, which contains an algorithm that is designed using the same parameters that were used in the learning algorithm and classification algorithm to originally determine the biomarkers. The algorithm produces a diagnosis based upon the data received relating to each biomarker.

In yet other preferred embodiments, the markers are captured and tested using non-SELDI forrnats. In one example, the sample is collected from the patient. The biomarkers are captured on a substrate using other known means, e.g., antibodies to the markers. The markers are detected using methods known in the art, e.g., optical methods and refractive index. Examples of optical methods include detection of fluorescence, e.g., ELISA. Examples of refractive index include surface plasmon resonance. The results for the markers are then subjected to an algorithm, which may or may not require artificial intelligence. The algorithm produces a diagnosis based upon the data received relating to each biomarker.

In any of the above methods, the data from the sample may be fed directly from the detection means into a computer containing the diagnostic algorithm. Alternatively, the data obtained can be fed manually, or via an automated means, into a separate computer that contains the diagnostic algorithm.

Exemplary Markers of the invention are illustrated in Tables 1 through 5:

TABLE 1

| Panel 1* | | | | |
|---|---|---|---|---|
| M/Z (Da) | PCa vs. HC | Cutoff | Sensitivity | Specificity |
| 7808.10 | down regulated | 1.3 | 89.3% | 89.5% |
| 14576.4 | down regulated | −0.7 | 67.0% | 63.2% |
| 2061.78 | Up regulated | 0.7 | 53.4% | 57.9% |
| 7973.38 | down regulated | 0.35 | 78.6% | 78.9% |
| 6677.09 | Up regulated | 0.8 | 63.1% | 78.9% |
| 3935.56 | down regulated | 1.2 | 84.5% | 84.2% |

TABLE 2

| Panel 2* | | | | |
|---|---|---|---|---|
| M/Z (Da) | PCa vs. BPH | Cutoff | Sensitivity | Specificity |
| 60,958.10 | Up regulated | −1.5 | 76.7% | 53.3% |
| 5,148.55 | Up regulated | 0 | 63.1% | 68.3% |
| 5,860.66 | Up regulated | −0.5 | 79.6% | 56.7% |
| 28,097.80 | down regulated | −0.7 | 60.2% | 63.3% |
| 2,995.64 | Up regulated | 0 | 65.0% | 55.0% |
| 24,346.00 | Up regulated | −1.3 | 65.0% | 63.3% |
| 6,721.91 | down regulated | 1.3 | 62.1% | 68.3% |
| 5,998.69 | Up regulated | −0.1 | 66.0% | 61.7% |
| 6,158.52 | Up regulated | 0 | 57.3% | 75.0% |
| 55,784.60 | Up regulated | −1.7 | 63.1% | 73.3% |

TABLE 3

| Panel 3* | | | | |
|---|---|---|---|---|
| M/Z (Da) | PT3-4 vs. pT2 | Cutoff | Sensitivity | Specificity |
| 2540.31 | Up regulated | 0.7 | 86.7% | 86.5% |
| 8018.62 | down regulated | 0 | 70.0% | 94.6% |
| 4657.57 | down regulated | −0.2 | 80.0% | 81.1% |
| 14702.6 | down regulated | −0.5 | 70.0% | 73.0% |

*confidence levels for the molecular weights are molecular weight ± 0.3%.

TABLE 4

| Additional to Panel | | | | |
|---|---|---|---|---|
| 2 | | Cutoff | Sensitivity | Specificity |
| 2.68 KD | Down regulated | −0.5 | 59.35% | 57.14% |
| 3.16 KD | Up regulated | −0.1 | 71.54% | 20.41% |
| 10.3 KD | Up regulated | 0 | 65.04% | 53.06% |
| 10.8 KD | Down regulated | −0.9 | 67.48% | 42.86% |
| 12.7 KD | Down regulated | −1.3 | 50.41% | 57.14% |
| 17.9 KD | Down regulated | −0.7 | 58.54% | 61.22% |

TABLE 5

| Additional to Panel | | | | |
|---|---|---|---|---|
| 3 | | Cutoff | Sensitivity | Specificity |
| 2.79 KD | Up regulated | −0.8 | 46.58% | 36.73% |
| 3.32 KD | Down regulated | −1.7 | 34.25% | 53.06% |
| 4.29 KD | Up regulated | −0.1 | 64.38% | 46.94% |
| 15.9 KD | Up regulated | −0.15 | 46.58% | 69.39% |
| 16.1 KD | Up regulated | −0.7 | 68.49% | 42.86% |
| 16.3 KD | Up regulated | −1.1 | 56.16% | 57.14% |

Detection of Organ confined Cancer OC = 1
T - if not Organ Confined OC = 0

using training group's up/down regulation NOT testing

Accordingly, embodiments of the invention include methods for aiding a human cancer diagnosis, wherein the method comprises: (a) detecting at least one marker in a sample, wherein the marker is selected from any of the Marker in Tables 1 through 5; and (b) correlating the detection of the marker or markers with a probable diagnosis of human cancer. The correlation may take into account the amount of the marker or markers in the sample compared to a control amount of the marker or markers (up or down regulation of the marker or markers) (e.g., in normal subjects in whom human cancer is undetectable). The correlation may take into account the presence or absence of the markers in a test sample and the frequency of detection of the same markers in a control. The correlation may take into account both of such factors to facilitate determination of whether a subject has a human cancer or not.

Any suitable samples can be obtained from a subject to detect markers. Preferably, a sample is a blood serum sample from the subject. If desired, the sample can be prepared as described above to enhance detectability of the markers. For example, to increase the detectability of markers in Tables 1 through 5, a blood serum sample from the subject can be preferably fractionated by, e.g., Cibacron blue agarose chromatography and single stranded DNA affinity chromatography, anion exchange chromatography and the like. Sample preparations, such as pre-fractionation protocols, are optional and may not be necessary to enhance detectability of markers depending on the methods of detection used. For example, sample preparation may be unnecessary if antibodies that specifically bind markers are used to detect the presence of markers in a sample.

### VII. DIAGNOSIS OF SUBJECT AND DETERMINATION OF PROSTATE CANCER STATUS

Any biomarker, individually, is useful in aiding in the determination of prostate cancer status. First, the selected biomarker is measured in a subject sample using the methods described herein, e.g., capture on a SELDI biochip followed by detection by mass spectrometry. Then, the measurement is compared with a diagnostic amount or control that distinguishes a prostate cancer status from a non-cancer status. The diagnostic amount will reflect the information herein that a particular biomarker is up-regulated or down-regulated in a cancer status compared with a non-cancer status. As is well understood in the art, the particular diagnostic amount used can be adjusted to increase sensitivity or specificity of the diagnostic assay depending on the preference of the diagnostician. The test amount as compared with the diagnostic amount thus indicates prostate cancer status.

While individual biomarkers are useful diagnostic markers, it has been found that a combination of biomarkers provides greater predictive value than single markers alone. Specifically, the detection of a plurality of markers in a sample increases the percentage of true positive and true negative diagnoses and would decrease the percentage of false positive or false negative diagnoses. Thus, preferred methods of the present invention comprise the measurement of more than one biomarker. For example, the methods of the present invention have an AUC from ROC analysis greater than 0.50, more preferred methods have an AUC greater than 0.60, more preferred methods have an AUC greater than 0.70. Especially preferred methods have an AUC greater than 0.70 and most preferred methods have an AUC greater than 0.80.

In order to use the biomarkers in combination, a logistical regression algorithm is useful. The UMSA algorithm is particularly useful to generate a diagnostic algorithm from test data. This algorithm is disclosed in Z. Zhang et al., Applying classification separability analysis to microaary data. In: Lin S M, Johnson K F, eds. Methods of Microarray data analysis: papers from CAMDA '00. Boston: Kluwer Academic Publishers, 2001:125-136; and Z. Zhang et al., Fishing Expedition—a Supervised Approach to Extract Patterns from a Compendium of Expression Profiles. In Lin S M, Johnson, K F, eds. Microarray Data Analysis II: Papers from CAMDA '01. Boston: Kluwer Academic Publishers, 2002.

The learning algorithm will generate a multivariate classification (diagnostic) algorithm tuned to the particular specificity and sensitivity desired by the operator. The classification algorithm can then be used to determine prostate cancer status. The method also involves measuring the selected biomarkers in a subject sample. These measurements are submitted to the classification algorithm. The classification algorithm generates an indicator score that indicates cancer status.

The detection of the marker or markers is then correlated with a probable diagnosis of human cancer. In some embodiments, the detection of the mere presence or absence of a marker, without quantifying the amount of marker, is useful and can be correlated with a probable diagnosis of human cancer. For example, markers in Tables 1 through 3 can be more frequently detected in human prostate cancer patients than in normal subjects. The markers also differentiated between the different disease states, for example, prostate cancer (PCa) from healthy men as control (HC), PCa from benign prostatic hyperplasia (BPH), and non-organ confined PCa (pT3-4) from organ confined PCa (pT2), respectively and any combinations thereof. Thus, a mere detection of one or more of these markers in a subject being tested indicates that the subject has a higher probability of having a human cancer.

In other embodiments, the measurement of markers can involve quantifying the markers to correlate the detection of markers with a probable diagnosis of cancer. Thus, if the amount of the markers detected in a subject being tested is different compared to a control amount (i.e., higher or lower than the control, depending on the marker), then the subject being tested has a higher probability of having cancer.

The correlation may take into account the amount of the marker or markers in the sample compared to a control amount of the marker or markers (up or down regulation of the marker or markers) (e.g., in normal subjects in whom human cancer is undetectable). A control can be, e.g., the average or median amount of marker present in comparable samples of normal subjects in whom human cancer is undetectable. The control amount is measured under the same or substantially similar experimental conditions as in measuring the test amount. The correlation may take into account the presence or absence of the markers in a test sample and the frequency of detection of the same markers in a control. The correlation may take into account both of such factors to facilitate determination of prostate cancer status.

In certain embodiments of the methods of qualifying cancer status, the methods further comprise managing subject treatment based on the status. As aforesaid, such management describes the actions of the physician or clinician subsequent to determining cancer status. For example, if the result of the methods of the present invention is inconclusive or there is reason that confirmation of status is necessary, the physician may order more tests. Alternatively, if the status indicates that surgery is appropriate, the physician may schedule the patient for surgery. In other instances, the patient may receive chemotherapy or radiation treatments, either in lieu of, or in addition to, surgery. Likewise, if the result is negative, e.g., the status indicates late stage cancer or if the status is otherwise acute, no further action may be warranted. Furthermore, if the results show that treatment has been successful, no further management may be necessary.

The invention also provides for such methods where the biomarkers (or specific combination of biomarkers) are measured again after subject management. In these cases, the methods are used to monitor the status of the cancer, e.g., response to cancer treatment, remission of the disease or progression of the disease. Because of the ease of use of the methods and the lack of invasiveness of the methods, the methods can be repeated after each treatment the patient receives. This allows the physician to follow the effectiveness of the course of treatment. If the results show that the treatment is not effective, the course of treatment can be altered accordingly. This enables the physician to be flexible in the treatment options.

In another example, the methods for detecting markers can be used to assay for and to identify compounds that modulate expression of these markers in vivo or in vitro.

The methods of the present invention have other applications as well. For example, the markers can be used to screen for compounds that modulate the expression of the markers in vitro or in vivo, which compounds in turn may be useful in treating or preventing cancer in patients. In another example, the markers can be used to monitor the response to treatments for cancer. In yet another example, the markers can be used in heredity studies to determine if the subject is at risk for developing cancer. For instance, certain markers may be genetically linked. This can be determined by, e.g., analyzing samples from a population of prostate cancer patients whose families have a history of prostate cancer. The results can then be compared with data obtained from, e.g., cancer patients whose families do not have a history of prostate cancer. The markers that are genetically linked may be used as a tool to determine if a subject whose family has a history of prostate cancer is pre-disposed to having prostate cancer.

### VIII. KITS

In yet another aspect, the invention provides kits for aiding a diagnosis of human cancer, wherein the kits can be used to detect the markers of the present invention. For example, the kits can be used to detect any one or more of the markers described herein, which markers are differentially present in samples of a human cancer patient and normal subjects. The kits of the invention have many applications. For example, the kits can be used to differentiate if a subject has human prostate cancer or has a negative diagnosis, thus aiding a human cancer diagnosis. In another example, the kits can be used to identify compounds that modulate expression of one or more of the markers in in vitro or in vivo animal models for human cancer.

In one embodiment, a kit comprises: (a) a substrate comprising an adsorbent thereon, wherein the adsorbent is suitable for binding a marker, and (b) instructions to detect the marker or markers by contacting a sample with the adsorbent and detecting the marker or markers retained by the adsorbent. In some embodiments, the kit may comprise an eluant (as an alternative or in combination with instructions) or instructions for making an eluant, wherein the combination of the adsorbent and the eluant allows detection of the markers using gas phase ion spectrometry. Such kits can be prepared from the materials described above, and the previous discussion of these materials (e.g., probe substrates, adsorbents, washing solutions, etc.) is fully applicable to this section and will not be repeated.

In another embodiment, the kit may comprise a first substrate comprising an adsorbent thereon (e.g., a particle functionalized with an adsorbent) and a second substrate onto which the first substrate can be positioned to form a probe which is removably insertable into a gas phase ion spectrometer. In other embodiments, the kit may comprise a single substrate which is in the forrn of a removably insertable probe with adsorbents on the substrate. In yet another embodiment, the kit may further comprise a pre-fractionation spin column (e.g., Cibacron blue agarose column, anti-HSA agarose column, K-30 size exclusion column, Q-anion exchange spin column, single stranded DNA column, lectin column, etc.).

Optionally, the kit can further comprise instructions for suitable operational parameters in the form of a label or a separate insert. For example, the kit may have standard instructions informing a consumer how to wash the probe after a sample of blood serum is contacted on the probe. In another example, the kit may have instructions for pre-fractionating a sample to reduce complexity of proteins in the sample. In another example, the kit may have instructions for automating the fractionation or other processes.

In another embodiment, a kit comprises (a) an antibody that specifically binds to a marker; and (b) a detection reagent. Such kits can be prepared from the materials described above, and the previous discussion regarding the materials (e.g., antibodies, detection reagents, immobilized supports, etc.) is fully applicable to this section and will not be repeated. Optionally, the kit may further comprise pre-fractionation spin columns. In some embodiments, the kit may further comprise instructions for suitable operation parameters in the form of a label or a separate insert.

Optionally, the kit may further comprise a standard or control information so that the test sample can be compared with the control information standard to determine if the test amount of a marker detected in a sample is a diagnostic amount consistent with a diagnosis of human prostate cancer.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### EXAMPLES

Materials and Methods

SELDI

Surface enhanced-laser desorption/ionization is an affinity-based mass spectrometry method in which proteins of interest are selectively adsorbed to a chemically modified surface on the chip. Other substances are removed by washing steps.

### Example 1

### Study Groups

Healthy men (HC) as control (19, PSA<4 ng/ml), histologically confirmed BPH (60), organ confined cancer (pT2, 37), non-organ confined cancer (pT3 and pT4, 30) obtained preoperatively, and men with biopsy-proven cancer and unknown pathologic stage (36). Serum samples (0.5 ul) were applied to the IMAC3-Ni²⁺ chips. Proteins bound to the chip surface were analyzed by a PBS-II mass reader. A total of 138 peaks were selected using Ciphergen ProteinChip Software3.0 (Ciphergen, Calif.). The software package ProPeak (3Z Informatics, SC) was used to compute a linear combination of selected mass spectrum peaks that offers the optimal separation between a given pair of diagnostic groups based on the Unified Maximum Separability Analysis (UMSA) algorithm. The result is a single-valued predictive index. Sensitivity and specificity were calculated based on a selected cutoff value.

Differentiation results between prostate disease and non-disease groups by SELDI protein chip/mass spectrometry are summarized in the Tables which shows the results for each individual panel of biomarkers. It should be noted that for comparison between different pairs of diagnostic groups, the selection and combination of peaks could be different. The high sensitivity and specificity achieved by serum protein profiling indicate that SELDI-protein chip/mass spectrometry combined with bioinformatics tools can greatly facilitate the discovery of new and better biomarkers for prostate diseases.

TABLE 1
Differentiation between prostate disease and non-disease groups by
SELDI protein chip/mass spectrometry.
A. Performance of a combination of selected top biomarkers:

| Disease/non- | Differentiation Between Diagnostic Groups | | |
|---|---|---|---|
| disease | Ca/HC | Ca/BPH | pT3-4/pT2 |
| Sensitivity | 92% | 80% | 90% |
| | (95/103) | (82/103) | (27/30) |
| Specificity | 90% | 77% | 95% |
| | (17/19) | (46/60) | (35/37) |
| Peaks (M/Z)^{a} | Top 6 | Top 10 | Top 4 |

B. Performance of individual biomarkers:
Panel 1

| M/Z (Da)^{a} | PCa vs. HC | Cutoff | Sensitivity | Specificity |
|---|---|---|---|---|
| 7808.10 | down regulated | 1.3 | 89.3% | 89.5% |
| 14576.4 | down regulated | −0.7 | 67.0% | 63.2% |
| 2061.78 | Up regulated | 0.7 | 53.4% | 57.9% |
| 7973.38 | down regulated | 0.35 | 78.6% | 78.9% |
| 6677.09 | Up regulated | 0.8 | 63.1% | 78.9% |
| 3935.56 | down regulated | 1.2 | 84.5% | 84.2% |

Panel 2

| M/Z (Da)^{a} | PCa vs. BPH | Cutoff | Sensitivity | Specificity |
|---|---|---|---|---|
| 60,958.10 | Up regulated | −1.5 | 76.7% | 53.3% |
| 5,148.55 | Up regulated | 0 | 63.1% | 68.3% |
| 5,860.66 | Up regulated | −0.5 | 79.6% | 56.7% |
| 28,097.80 | down regulated | −0.7 | 60.2% | 63.3% |
| 2,995.64 | Up regulated | 0 | 65.0% | 55.0% |
| 24,346.00 | Up regulated | −1.3 | 65.0% | 63.3% |
| 6,721.91 | down regulated | 1.3 | 62.1% | 68.3% |
| 5,998.69 | Up regulated | −0.1 | 66.0% | 61.7% |
| 6,158.52 | Up regulated | 0 | 57.3% | 75.0% |
| 55,784.60 | Up regulated | −1.7 | 63.1% | 73.3% |

Panel 3

| M/Z (Da)^{a} | PT3-4 vs. pT2 | Cutoff | Sensitivity | Specificity |
|---|---|---|---|---|
| 2540.31 | Up regulated | 0.7 | 86.7% | 86.5% |
| 8018.62 | down regulated | 0 | 70.0% | 94.6% |
| 4657.57 | down regulated | −0.2 | 80.0% | 81.1% |
| 14702.6 | down regulated | −0.5 | 70.0% | 73.0% |

^{a}M/Z (mass-dependent velocities)

### Example 2

A total of 343 men who had an archival serum sample available were included in this study. The cancer group consisted of 244 men underwent radical prostatectomy at Johns Hopkins Hospital between March 1999 and April 2001, 146 cases with organ-confined and 98 with non-confined disease at pathologic examination. The non-cancer group included 99 men diagnosed with benign prostate diseases between April 1997 and April 2001 at the same institution. Serum proteomic mass spectra of these patients were generated using IMAC-Ni ProteinChip® arrays and a PBS-II laser desorption/ionization time-of-flight mass spectrometer. The entire sample set was randomly divided into training and testing. The biomarkers were selected using the training data and the sensitivity, specificity estimated using the testing data set.

Differentiation results between prostate disease and non-disease groups by SELDI protein chip/mass spectrometry are summarized in the Tables which shows the results for each individual panel of biomarkers. It should be noted that for comparison between different pairs of diagnostic groups, the selection and combination of peaks could be different.

Performance of individual biomarkers:

| | | |
|---|---|---|
| Cutoff | Sensitivity | Specificity |

Additional markers - Panel 2

| | | | | |
|---|---|---|---|---|
| 2.68 KD | Down regulated | −0.5 | 59.35% | 57.14% |
| 3.16 KD | Up regulated | −0.1 | 71.54% | 20.41% |
| 10.3 KD | Up regulated | 0 | 65.04% | 53.06% |
| 10.8 KD | Down regulated | −0.9 | 67.48% | 42.86% |
| 12.7 KD | Down regulated | −1.3 | 50.41% | 57.14% |
| 17.9 KD | Down regulated | −0.7 | 58.54% | 61.22% |

Additional markers - Panel 3

| | | | | |
|---|---|---|---|---|
| 2.79 KD | Up regulated | −0.8 | 46.58% | 36.73% |
| 3.32 KD | Down regulated | −1.7 | 34.25% | 53.06% |
| 4.29 KD | Up regulated | −0.1 | 64.38% | 46.94% |
| 15.9 KD | Up regulated | −0.15 | 46.58% | 69.39% |
| 16.1 KD | Up regulated | −0.7 | 68.49% | 42.86% |
| 16.3 KD | Up regulated | −1.1 | 56.16% | 57.14% |

Detection of Organ confined Cancer OC = 1
T - if not OrganConfined OC = 0
using training group's up/down regulation NOT testing

The present invention has been described in detail, including the preferred embodiments thereof. However, it will be appreciated that those skilled in the art, upon consideration of the present disclosure, may make modifications and/or improvements of this invention and still be within the scope and spirit of this invention as set forth in the following claims.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

1. A method of qualifying prostate cancer status in a subject comprising:
(a) measuring at least one biomarker in a sample from the subject, wherein the biomarker is selected from the group consisting of
Marker I: having a molecular weight of about 7.808 kD
Marker II: having a molecular weight of about 14.576 kD
Marker III: having a molecular weight of about 2.062 kD
Marker IV: having a molecular weight of about 7.974 kD
Marker V: having a molecular weight of about 6.677 kD
Marker VI: having a molecular weight of about 3.936 kD
Marker VII: having a molecular weight of about 60.958 kD
Marker VIII: having a molecular weight of about 5.149 kD
Marker IX: having a molecular weight of about 5.861 kD
Marker X: having a molecular weight of about 28.098 kD
Marker XI: having a molecular weight of about 2.996 kD
Marker XII: having a molecular weight of about 24.346 kD
Marker XIII: having a molecular weight of about 6.722 kD
Marker XIV: having a molecular weight of about 5.999 kD
Marker XV: having a molecular weight of about 6.158 kD
Marker XVI: having a molecular weight of about 55.785 kD
Marker XVII: having a molecular weight of about 2.540 kD
Marker XVIII: having a molecular weight of about 8.019 kD
Marker XIX: having a molecular weight of about 4.658 kD
Marker XX: having a molecular weight of about 14.703 kD
Marker XXI: having a molecular weight of about 2.68 kD
Marker XXII: having a molecular weight of about 3.16 kD
Marker XXIII: having a molecular weight of about 10.3 kD
Marker XXIV: having a molecular weight of about 10.8 kD
Marker XXV: having a molecular weight of about 12.7 kD
Marker XXVI: having a molecular weight of about 17.9 kD
Marker XXVII: having a molecular weight of about 2.79 kD
Marker XXVIII: having a molecular weight of about 3.32 kD
Marker XXIX: having a molecular weight of about 4.29 kD
Marker XXX: having a molecular weight of about 15.9 kD
Marker XXXI: having a molecular weight of about 16.1 kD
Marker XXXII: having a molecular weight of about 16.3 kD, and combinations thereof, and
(b) correlating the measurement with prostate cancer status.

2. The method of claim 1 further comprising:
(c) managing subject treatment based on the status.

3. The method of claim 2, wherein managing subject treatment is selected from ordering more tests, performing surgery, and taking no further action.

4. The method of claim 2 further comprising:
(d) measuring the at least one biomarker after subject management.

5. The method of claim 1 wherein the prostate cancer status is selected from the group consisting of the subject's risk of cancer, the presence or absence of disease, the type of disease, the stage of disease and the effectiveness of treatment of disease.

6. A method for differentiating between a diagnosis of prostate cancer and non-prostate cancer comprising:
(a) detecting in a subject sample an amount of at least one biomarker selected from the group consisting of:
Marker I: having a molecular weight of about 7.808 kD
Marker II: having a molecular weight of about 14.576 kD
Marker III: having a molecular weight of about 2.061 kD
Marker IV: having a molecular weight of about 7.973 kD
Marker V: having a molecular weight of about 6.677 kD and
Marker VI: having a molecular weight of about 3.935 kD; and
(b) correlating the amount with a diagnosis of prostate cancer or non-prostate cancer.

7. A method for differentiating between a diagnosis of prostate cancer and benign prostate hyperplasia comprising:
(a) detecting in a subject sample an amount of at least one biomarker selected from the group consisting of:
Marker VII: having a molecular weight of about 60.958 kD
Marker VIII: having a molecular weight of about 5.148 kD
Marker IX: having a molecular weight of about 5.860 kD
Marker X: having a molecular weight of about 28.097 kD
Marker XI: having a molecular weight of about 2.996 kD
Marker XII: having a molecular weight of about 24.346 kD
Marker XIII: having a molecular weight of about 6.722 kD
Marker XIV: having a molecular weight of about 5.999 kD
Marker XV: having a molecular weight of about 6.159 kD
Marker XVI: having a molecular weight of about 55.785 kD
(b) correlating the amount with a diagnosis of prostate cancer or benign prostate hyperplasia.

8. A method for differentiating between a diagnosis of prostate cancer and benign prostate hyperplasia comprising:
(a) detecting in a subject sample an amount of at least one biomarker selected from the group consisting of:
Marker XXI: having a molecular weight of about 2.68 kD
Marker XXII: having a molecular weight of about 3.16 kD
Marker XXIII: having a molecular weight of about 10.3 kD
Marker XXIV: having a molecular weight of about 10.8 kD
Marker XXV: having a molecular weight of about 12.7 kD, and
Marker XXVI: having a molecular weight of about 17.9 kD.
(b) correlating the amount with a diagnosis of prostate cancer or benign prostate hyperplasia.

9. A method for differentiating between a diagnosis of organ defined prostate cancer and non-organ-defined prostate cancer comprising:
(a) detecting in a subject sample an amount of at least one biomarker selected from the group consisting of:
Marker XVII: having a molecular weight of about 2.540 kD
Marker XVIII: having a molecular weight of about 8.018 kD
Marker XIX: having a molecular weight of about 4.658kD,
and
Marker XX: having a molecular weight of about 14.703 kD; and
(b) correlating the amount with a diagnosis of organ-defined prostate cancer or non-organ-defined prostate cancer.

10. A method for differentiating between a diagnosis of organ defined prostate cancer and non-organ-defined prostate cancer comprising:
(a) detecting in a subject sample an amount of at least one biomarker selected from the group consisting of:
Marker XXVII: having a molecular weight of about 2.79 kD
Marker XXVIII: having a molecular weight of about 3.32 kD
Marker XXIX: having a molecular weight of about 4.29 kD
Marker XXX: having a molecular weight of about 15.9 kD
Marker XXXI: having a molecular weight of about 16.1 kD, and
Marker XXXII: having a molecular weight of about 16.3 kD.
(b) correlating the amount with a diagnosis of organ-defined prostate cancer or non-organ-defined prostate cancer.

11. The method of claim 1 wherein the marker is detected by mass spectrometry.

12. The method of claim 1 wherein the marker is detected by capturing the marker on a biochip having an affinity surface and detecting the captured marker by SELDI.

13-29. (canceled)

30. A kit for aiding the diagnosis of cancer, comprising:
an adsorbent attached to a substrate, wherein the adsorbent retains one or more biomarkers selected from:
Marker I: having a molecular weight of about 7.808 kD
Marker II: having a molecular weight of about 14.576 kD
Marker III: having a molecular weight of about 2.062 kD
Marker IV: having a molecular weight of about 7.974 kD
Marker V: having a molecular weight of about 6.677 kD
Marker VI: having a molecular weight of about 3.936 kD
Marker VII: having a molecular weight of about 60.958 kD
Marker VIII: having a molecular weight of about 5.149 kD
Marker IX: having a molecular weight of about 5.861 kD
Marker X: having a molecular weight of about 28.098 kD
Marker XI: having a molecular weight of about 2.996 kD
Marker XII: having a molecular weight of about 24.346 kD
Marker XIII: having a molecular weight of about 6.722 kD
Marker XIV: having a molecular weight of about 5.999 kD
Marker XV: having a molecular weight of about 6.159 kD
Marker XVI: having a molecular weight of about 55.784 kD
Marker XVII: having a molecular weight of about 2.540 kD
Marker XVIII: having a molecular weight of about 8.019 kD
Marker XIX: having a molecular weight of about 4.658 kD
Marker XX: having a molecular weight of about 14.703 kD
Marker XXI: having a molecular weight of about 2.68 kD
Marker XXII: having a molecular weight of about 3.16 kD
Marker XXIII: having a molecular weight of about 10.3 kD
Marker XXIV: having a molecular weight of about 10.8 kD
Marker XXV: having a molecular weight of about 12.7 kD
Marker XXVI: having a molecular weight of about 17.9 kD
Marker XXVII: having a molecular weight of about 2.79 kD
Marker XXVIII: having a molecular weight of about 3.32 kD
Marker XXIX: having a molecular weight of about 4.29 kD
Marker XXX: having a molecular weight of about 15.9 kD
Marker XXXI: having a molecular weight of about 16.1 kD, and
Marker XXXII: having a molecular weight of about 16.3 kD.

31. The kit of claim 30, further comprising written instructions for use of the kit for detection of cancer.

32-41. (canceled)

42. A protein purified on a biochip selected from:
Marker I: having a molecular weight of about 7.808 kD
Marker II: having a molecular weight of about 14.576 kD
Marker III: having a molecular weight of about 2.062 kD
Marker IV: having a molecular weight of about 7.974 kD
Marker V: having a molecular weight of about 6.667 kD
Marker VI: having a molecular weight of about 3.936 kD
Marker VII: having a molecular weight of about 60.958 kD
Marker VIII: having a molecular weight of about 5.149 kD
Marker IX: having a molecular weight of about 5.861 kD
Marker X: having a molecular weight of about 28.098 kD
Marker XI: having a molecular weight of about 2.996 kD
Marker XII: having a molecular weight of about 24.346 kD
Marker XIII: having a molecular weight of about 6.722 kD
Marker XIV: having a molecular weight of about 5.999 kD
Marker XV: having a molecular weight of about 6.159 kD
Marker XVI: having a molecular weight of about 55.784 kD
Marker XVII: having a molecular weight of about 2.540 kD
Marker XVIII: having a molecular weight of about 8.019 kD
Marker XIX: having a molecular weight of about 4.658 kD
Marker XX: having a molecular weight of about 14.703 kD
Marker XXI: having a molecular weight of about 2.68 kD
Marker XXII: having a molecular weight of about 3.16 kD
Marker XXIII: having a molecular weight of about 10.3 kD
Marker XXIV: having a molecular weight of about 10.8 kD
Marker XXV: having a molecular weight of about 12.7 kD
Marker XXVI: having a molecular weight of about 17.9 kD
Marker XXVII: having a molecular weight of about 2.79 kD
Marker XXVIII: having a molecular weight of about 3.32 kD
Marker XXIX: having a molecular weight of about 4.29 kD
Marker XXX: having a molecular weight of about 15.9 kD
Marker XXXI: having a molecular weight of about 16.1 kD, and
Marker XXXII: having a molecular weight of about 16.3 Kd.

43. The purified proteins of claim 42, comprising a composition of a combination of at least two proteins.

44-77. (canceled)
